(19) United States
(12) Patent Application Publication
(10) Pub. No.: US 2006/0058380 A1
(43) Pub. Date: Mar. 16, 2006

(54) Novel aminobenzophenone compounds

(76) Inventors: Erik Rytter Ottosen, Olstykke (DK); Bjorkling Fredrik, Helsingborg (SE); Heinz Wilhelm Dannacher, Skovlunde (DK)

Correspondence Address: BIRCH STEWART KOLASCH & BIRCH, PO BOX 747, FALLS CHURCH, VA, 22040-0747, US

(21) Appl. No.: 10/539,602

(22) PCT Filed: Dec. 19, 2003
(86) PCT No.: PCT/DK03/00900

§ 371 Date: Jun. 17, 2005

Related U.S. Application Data
(60) Provisional application No. 60/434,798, filed on Dec. 20, 2002.

(51) Int. Cl. A61K 31/325; C07C 271/26

(52) U.S. Cl. 514/487; 560/27; 558/410

(57) ABSTRACT

The invention provides novel compounds according to formula I relates to compounds with the general formula I 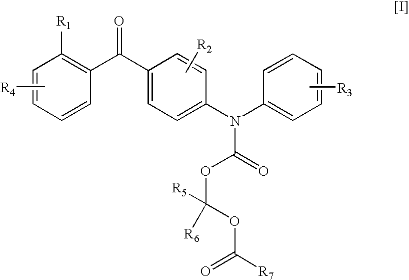 said compounds being useful, e.g. in the treatment of inflammatory diseases.

### FIELD OF THE INVENTION

The invention relates to a novel class of aminobenzophenones and to their use in therapy.

### BACKGROUND OF THE INVENTION

Aminobenzophenones are well-described in the scientific as well as patent literature. WO 98/32730, WO 01/05746, WO 01/05749, WO 01/05751 and WO 01/05745 all disclose compounds with the common core-structure 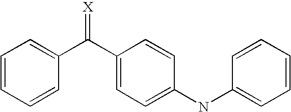
wherein the phenyl ring to the right is substituted by an amine. Moreover, WO 01/42189 and WO 02/076447 disclose compounds with a similar structure, but with no amine substituent in the phenyl ring to the right. Finally, WO 01/90074 and WO 02/083622 disclose compounds where the right-most and left-most phenyl rings, respectively are replaced by heterocycles. The compounds of these patent applications are indicated to be effective inhibitors of interleukin 1β (IL-1β) and tumour necrosis factor α (TNF-α) secretion in vitro, said compounds being potentially useful for treatment of inflammatory diseases in which the production of cytokines is involved in the pathogenesis. Apparently, aminobenzophenones exert their effect by an inhibition of p38 MAP kinase, which in turn inhibits the production of IL-1β and TNF-α.

The preparation of structurally related aminobenzophenones useful as dyes for textiles is disclosed in Man-Made Text. India (1987), 30(6), 275-6, Man-Made Text. India (1986), 29(5), 224-30, and Man-Made Text. India (1985), 28(11), 425, 427-9, 431.

It has, however, been found that known aminobenzophenones discolour when exposed to light, probably due to the presence of aromatic amines in a highly conjugated environment. Hence, when the compounds are applied to the skin, the skin darkens into a yellow or even blackish shade. This is, of course, unacceptable in many situations, and at any rate, it severely restricts the applicability of aminobenzophenones for treatment of dermal diseases or states.

It is therefore the purpose of the present invention to provide aminobenzophenones which do not discolour when exposed to light, and which thus lend them selves more readily to dermal applications.

### SUMMARY OF THE INVENTION

Accordingly, the present invention relates to compounds of general formula I 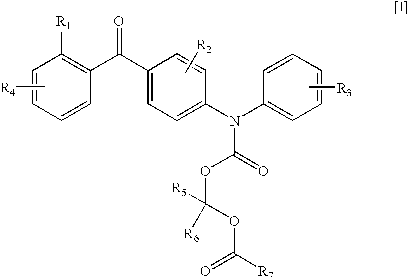
wherein R₁ represents a substituent selected from the group consisting of halogen, hydroxy, mercapto, trifluoromethyl, amino, (C₁-C₃)alkyl, (C₂-C₃)olefinic group, (C₁-C₃)alkoxy, (C₁-C₃)alkylthio, (C₁-C₄)alkylamino and cyano;
R₂ represents one or more, same or different substituents selected from the group consisting of hydrogen, halogen, hydroxy, mercapto, trifluoromethyl, amino, (C₁-C₃)alkyl, (C₂-C₃)olefinic group, (C₁-C₃)alkoxy, (C₁-C₃)alkylthio, (C₁-C₄)alkylamino, (C₁-C₃)alkoxycarbonyl, cyano, and nitro;
R₃ represents one or more, same or different substituents selected from the group consisting of hydrogen, halogen, hydroxy, mercapto, trifluoromethyl, cyano, carboxy, carbamoyl, (C₁-C₄)alkyl, (C₂-C₄)olefinic group, (C₁-C₄)alkoxy, (C₁-C₄)alkylthio, and (C₁-C₄)alkoxycarbonyl;
R₄ represents one or more, same or different substituents selected from the group consisting of hydrogen, halogen, hydroxy, mercapto, trifluoromethyl, amino, (C₁-C₃)alkyl, (C₂-C₃)olefinic group, (C₁-C₃)alkoxy, (C₁-C₃)alkylthio, (C₁-C₄)alkylamino, (C₁-C₃)alkoxycarbonyl, cyano, and nitro;
R₅ represents hydrogen, (C₁-C₆)alkyl and (C₂-C₆)olefinic group;
R₆ represents hydrogen, (C₁-C₆)alkyl and (C₂-C₆)olefinic group;
R₇ represents (C₁-C₁₈)alkyl, (C₃-C₈)cyclic hydrocarbon group, (C₂-C₁₈ )olefinic group, heterocyclyl, (C₂-C₁₈)alkynyl, (C₁-C₁₈)alkyl-heterocyclyl, (C₁-C₁₈)alkyl-(C₃-C₈)cyclic hydrocarbon group, (C₂-C₁₈)olefinic group-heterocyclyl, (C₂-C₁₈)olefinic group-(C₃-C₈)cyclic hydrocarbon group, (C₂-C₁₈)alkynyl-heterocyclyl, (C₂-C₁₈)alkynyl-(C₃-C₈)cyclic hydrocarbon group; and wherein R₇ may optionally be substituted by one or more substituents represented by R₈;
R₈ represents halogen, hydroxy, mercapto, trifluoromethyl, amino, (C₁-C₆)alkyl, (C₁-C₆)alkoxy, (C₁-C₆)alkylthio, (C₁-C₆)alkylamino, (C₁-C₆)alkoxycarbonyl, (C₁-C₉)trialkylammonium in association with a pharmaceutically acceptable anion, (C₂-C₁₂)dialkylphosphinoyl, (C₁-C₆)alkyl(hydroxy)phosphinoyl, (C₂-C₁₂)dialkylphosphinoyloxy, (C₁-C₆)alkyl(hydroxy)phosphinoyloxy, dihydroxyphosphinoyl, dihydroxyphosphinoyloxy, cyano, azido, nitro, —CHO, —COOH, —CONH₂, —CONHR′, —CONRR′ wherein R and R′ represent (C₁-C₃)alkyl or Y—R₉;
Y represents —O—, —S—, —S(O)—, —S(O)₂—, —NRₐ—, —NRₐC(O)NR_{b}—, —NRₐC(O)—, —C(O)NRₐ—, —C(O)—, —C(O)O—, —OC(O)—, —NRₐC(O)O—, —OC(O)NRₐ—, —S(O)₂NRₐ—, —NRₐS(O)₂—, —OC(O)O— or —O(CH₂CH₂O)ₙ— wherein n is an integer between 1 and 6, and Rₐ and R_{b} independently represents hydrogen or (C₁-C₃)alkyl;
R₉ represents (C₁-C₆ )alkyl, (C₂-C₆)olefinic group, (C₃-C₆)cyclic hydrocarbon group, heterocyclyl, (C₂-C₆)alkynyl, (C₁-C₆)alkyl-(C₃-C₆)cyclic hydrocarbon or (C₁-C₆)alkyl-heterocyclyl, and wherein R₉ may optionally be substituted by one or more substituents represented by R₁₀;
R₁₀ represents halogen, hydroxy, mercapto, trifluoromethyl, amino, (C₁-C₆)alkyl, (C₁-C₆)alkoxy, (C₁-C₆)alkylthio, (C₁-C₆)alkylamino or (C₁-C₆)alkoxycarbonyl;
and pharmaceutically acceptable salts, solvates and hydrates thereof.

The invention also relates to compounds of formula I for use in therapy, and in particular to pharmaceutical compositions comprising a compound of formula I.

In a further embodiment, the invention relates to methods of treatment, the methods comprising administering to a patient in need thereof an effective amount of a compound of formula I.

In a yet further embodiment, the invention relates to the use of compounds of formula I in the manufacture of medicaments.

The compounds of formula I are prodrugs, as disclosed in WO 91/10639, in the sense that the moiety attached to the N-atom, i.e. 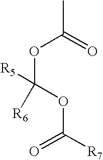
is cleaved, probably enzymatically, from the aminobenzophenone core once the compound has penetrated into the skin. In this way, the active, potentially colour generating compound is only formed when it is inside the skin, protected from light. The potentially colour generating compound is not exposed to light, and will therefore not give rise to a discolouration of the skin, while the active compound is still delivered to the affected part of the skin.

### DETAILED DESCRIPTION OF THE INVENTION

Compounds of formula I may comprise chiral carbon atoms and carbon-carbon double bonds which may give rise to the existence of isomeric forms, e.g. enantiomers, diastereomers and geometric isomers. The present invention relates to all such isomers, either in pure form or as mixtures thereof. Pure stereoisomeric forms of the compounds and the intermediates of this invention may be obtained by the application of art-known procedures. Diastereomers may be separated by physical separation methods such as selective crystallization and chromatographic techniques, e. g. liquid chromatography using chiral stationary phases. Enantiomers may be separated from each other by the selective crystallization of their diastereomeric salts with optically active acids. Alternatively, enantiomers may be separated by chromatographic techniques using chiral stationary phases. Said pure stereoisomeric forms may also be derived from the corresponding pure stereoisomeric forms of the appropriate starting materials, provided that the reaction occurs stereoselectively or stereospecifically. Preferably, if a specific stereoisomer is desired, said compound will be synthesized by stereoselective or stereospecific methods of preparation. These methods will advantageously employ chirally pure starting materials. Likewise, pure geometric isomers may be obtained from the corresponding pure geometric isomers of the appropriate starting materials. A mixture of geometric isomers will typically exhibit different physical properties, and they may thus be separated by standard chromatographic techniques well-known in the art.

The term “pharmaceutically acceptable salt” is intended to indicate salts prepared by reacting a compound of formula I with a suitable inorganic or organic acid, e.g. hydrochloric, hydrobromic, hydroiodic, sulfuric, nitric, acetic, phosphoric, lactic, maleic, phthalic, citric, propionic, benzoic, glutaric, gluconic, methanesulfonic, salicylic, succinic, tartaric, toluenesulfonic, sulfamic or fumaric acid. Pharmaceutically acceptable salts of compounds of formula I may also be prepared by reaction with a suitable base such as sodium hydroxide, potassium hydroxide, ammonia or the like.

The term “solvate” is intended to indicate a species formed by interaction between a compound, e.g. a compound of formula I, and a solvent, e.g. alcohol, glycerol and water, wherein said species are in a solid form. When water is the solvent, said species is referred to as a hydrate.

The term “halogen” is intended to indicate members of the seventh main group of the periodic table, i.e. fluoro, chloro, bromo and iodo.

The term “alkyl” is intended to indicate a univalent radical derived from a straight or branched alkane by removal of a hydrogen atom from any carbon atom, and it includes the subclasses of primary, secondary and tertiary alkyl groups, including for example (C₁-C₁₈)alkyl, (C₁-C₆)alkyl, methyl, ethyl, n-propyl, isopropyl, n-butyl, isobutyl, sec-butyl, t-butyl, pentyl, hexyl, heptyl, decanyl, etc.

The term “olefinic group” is intended to indicate a straight or branched hydrocarbon radical having one or more carbon-carbon double bonds of either E or Z stereochemistry where applicable. The term includes, for example, (C₂-C₁₈)olefinic group, (C₂-C₆)olefinic group and (C₂-C₃) olefinic group, vinyl, allyl, 1-butenyl, 2-butenyl, and 2-methyl-2-propenyl, 2,4-pentenedienyl, etc.

The term “alkoxy” is intended to indicate a radical of the formula —OR, where R is alkyl as defined above, for example (C₁-C₁₈)alkoxy, (C₁-C₆)alkoxy, methoxy, ethoxy, n-propoxy, tert-butoxy, etc.

The term “alkylthio” is intended to indicate a radical of the formula —SR, where R is alkyl as defined above, for example (C₁-C₁₈)alkylthio, (C₁-C₆)alkylthio, methylthio, ethylthio, n-propylthio, 2-propylthio, etc.

The term “alkylamino” is intended to indicate a radical of the formula —NHR or —NR₂, where each R is alkyl as defined above and includes, for example, methylamino, dimethylamino, di-(n-propyl)amino, n-butyl(ethyl)amino, etc.

The term “alkoxycarbonyl” is intended to indicate a radical of the formula —COOR, where R is alkyl as defined above and includes methoxycarbonyl, ethoxycarbonyl, n-propoxycarbonyl, i-propoxycarbonyl, etc.

The term “cyclic hydrocarbon group” includes saturated and unsaturated, optionally fused bicyclic, hydrocarbon rings, such as (C₃-C₈)cycloalkyl, cyclopropyl, cyclopentyl, cyclohexyl, and cyclooctyl, (C₃-C₈)cycloalkene group, cycloprop-2-enyl, cyclobut-2-enyl, cyclopent-2-enyl, cyclohex-3-enyl, cycloocta-4-enyl, cyclohex-3,5-dienyl and phenyl.

The term “cyclic hydrocarbon group” also includes compounds as just defined wherein one or more ring —CH₂— fragments have been replaced by a —C(O)— fragment and /or an exo-cyclic carbon-carbon double bond, such as oxocyclohexyl, oxocyclopentyl, 4-oxo-1,2,3,4-tetrahydronaphtalen-1-yl, 1-oxo-1,2,3,4-tetrahydronaphtalen-1-yl, 2-oxocyclohex-3-en-1-yl and 2-oxocyclohex-1-en-1-yl, and

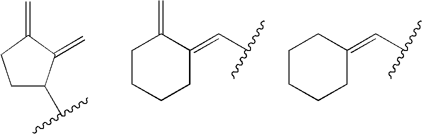

The term “alkynyl” is intended to indicate univalent group derived from a straight or branched alkyne by removal of a hydrogen atom from any carbon atom, and includes the subclasses of primary, secondary and tertiary alkyl groups respectively, and having the number of carbon atoms specified, including for example (C₁-C₁₈)alkynyl, (C₂-C₆)alkynyl, ethynyl, propynyl, 1,1-dimethyl-3-butynyl, etc.

The term “heterocyclyl” is intended to indicate saturated or unsaturated, optionally fused carbocyclic rings comprising one or more heteroatoms selected from the group consisting of O, N and S, such as pyrrolyl, furanyl, thiophenyl, imidazolyl, oxazolyl, thiazolyl, pyrazolyl, pyrrolidinyl, pyridinyl, pyrimidinyl, tetrahydrotiophenyl, tetrahydrofuranyl, tetrahydropyranyl, piperidinyl, putinyl, quinolinyl, isoquinolinyl, 1,2-dihydroquinolinyl, etc. The term “heterocyclyl” also includes compounds as just defined wherein one or more ring —CH₂— fragments have been replaced by a —C(O)— fragment and/or an exo-cyclic carbon-carbon double bond, such as dioxopiperidinyl, 1-oxo-3,4-dihydroisoquinolin-2(1H)-yl and

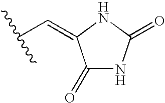

In a preferred embodiment, R₁ represents fluoro, chloro or bromo, methyl or methoxy, and particularly preferred in this embodiment, R₁ represents methyl.

In a preferred embodiment, R₂ represents on or more substituents selected from the Ist consisting of hydrogen, fluoro, chloro, methyl or methoxy, and particularly preferred in this embodiment, R₂ represents 2-chloro.

In a preferred embodiment, R₃ represents one or more substituents selected from the list consisting of hydrogen, fluoro, chloro, methyl, ethyl, ethenyl or methoxy, and particularly preferred in this embodiment, R₃ represents 2-methyl and 4-fluoro, or 2-methyl and 4-bromo.

In a preferred embodiment, R₄ represents one or more substituents selected from the list consisting of hydrogen, fluoro, chloro, bromo, methyl and methoxy, and particularly preferred in this embodiment, R₄ represents hydrogen or 4-chloro.

In a preferred embodiment, R₅ and R₆ each independently represent hydrogen or (C₁-C₆)alkyl, such as (C₁-C₄)alkyl, such as methyl.

In a preferred embodiment, R₇ represents (C₁-C₁₀)alkyl, (C₃-C₆)cyclic hydrocarbon group, (C₂-C₁₀)olefinic group, heterocyclyl, (C₂-C₁₀)alkynyl, (C₁-C₁₀)alkyl-heterocyclyl, (C₁-C₁₀)alkyl-(C₃-C₆)cyclic hydrocarbon group, (C₂-C₁₀)olefinic group-heterocyclyl, (C₂-C₁₀), olefinic group-(C₃-C₆)cyclic hydrocarbon group, (C₂-C₁₀)alkynyl-heterocyclyl, (C₂-C₁₀)alkynyl-(C₃-C₆)cyclic hydrocarbon group; and wherein R₇ may optionally be substituted by one or more substituents represented by R₈.

In a more preferred embodiment, R₇ represents (C₁-C₆)alkyl, (C₃-C₆)cyclic hydrocarbon group, (C₂-C₆)olefinic group, heterocyclyl, (C₂-C₆)alkynyl, (C₁-C₆)alkyl-heterocyclyl, (C₁-C₆)alkyl-(C₃-C₆)cyclic hydrocarbon group, (C₂-C₆)olefinic group-heterocyclyl, (C₂-C₆), olefinic group-(C₃-C₆)cyclic hydrocarbon group, (C₂-C₆)alkynyl-heterocyclyl, (C₂-C₆)alkynyl-(C₃-C₆)cyclic hydrocarbon group; and wherein R₇ may optionally be substituted by one or more substituents represented by R₈.

In particular, R₇ represents methyl, ethyl, propyl, iso-propyl, butyl, tert-butyl, pentyl, heptyl, nonyl, 2-methyl-propyl, 1-methyl-propyl, 2,2-dimethyl-propyl, cyclopropyl, cyclobutyl, phenyl, ethenyl, propenyl, phenylmethyl, phenyl-1-allyl or 2-, 3- or 4-pyridyl, all of which may be substituted by R₈.

In a preferred embodiment, R₈ represents halogen, hydroxy, trifluoromethyl, amino, (C₁-C₆)alkyl, (C₁-C₆)alkoxy, (C₁-C₆)alkylamino, (C₁-C₆)alkoxycarbonyl, (C₁-C₉)trialkylammonium in association with a pharmaceutically acceptable anion, cyano, COOH or Y—R₉.

In a preferred embodiment, R₈ represents hydroxyl or carboxy.

In a preferred embodiment, Y represents —O—, —NRₐ—, —NRₐC(O)—, —C(O)NRₐ—, —C(O)—, —C(O)O—, —OC(O)—, —NRₐC(O)O— or —O(CH₂CH₂O)ₙ— wherein n is 1, 2, 3 or 4, and Rₐ and R_{b} both represents hydrogen.

In a preferred embodiment, Y represents —C(O)—O—, NH—C(O)—O—, —O—, —O—C(O)— or — O(CH₂CH₂O)ₙ— wherein n is 3.

In a preferred embodiment, R₉ represents (C₁-C₄)alkyl, (C₂-C₃)olefinic group, (C₃-C₆)cyclic hydrocarbon group, heterocyclyl, (C₂-C₃)alkynyl, (C₁-C₃)alkyl-(C₃-C₆)cyclic hydrocarbon or (C₁-C₃)alkyl-heterocyclyl, wherein R₉ may optionally be substituted by one or more substituents represented by R₁₀.

In a preferred embodiment, R₉ represents (C₁-C₄)alkyl or (C₁-C₃)alkyl-(C₃-C₆)cyclic hydrocarbon, and particularly preferred in this embodiment, R₉ represents methyl, ethyl, tert-butyl or phenylmethyl.

In a preferred embodiment, R₁₀ represents fluoro, chloro, hydroxy, trifluoromethyl, amino, (C₁-C₃)alkyl, (C₁-C₃ )alkoxy, (C₁-C₃)alkylamino or (C₁-C₃)alkoxycarbonyl.

Another preferred embodiment of the present invention relates to compounds of formula I wherein R₁ is methyl; R₂ is 2-chloro; R₃ is 2-methyl and 4-fluoro, or 2-methyl and 4-bromo; R₄ is hydrogen or 4-chloro;
R₅ and R₆ independently represent hydrogen or (C₁-C₄)alkyl;
R₇ represents (C₁-C₁₀)alkyl, (C₃-C₆)cyclic hydrocarbon group, (C₂-C₁₀)olefinic group, heterocyclyl, (C₂-C₁₀)alkynyl, (C₁-C₁₀)alkyl-heterocyclyl, (C₁-C₁₀)alkyl-(C₃-C₆)cyclic hydrocarbon group, (C₂-C₁₀)olefinic group-heterocyclyl, (C₂-C₁₀), olefinic group-(C₃-C₆)cyclic hydrocarbon group, (C₂-C₁₀)alkynyl-heterocyclyl, (C₂-C₁₀)alkynyl-(C₃-C₆)cyclic hydrocarbon group; and wherein R₇ may optionally be substituted by one or more substituents represented by R₈;
R₈ represents halogen, hydroxy, trifluoromethyl, amino, (C₁-C₆)alkyl, (C₁-C₆)alkoxy, (C₁-C₆)alkylamino, (C₁-C₆ )alkoxycarbonyl, (C₁-C₉)trialkylammonium in association with a pharmaceutically acceptable anion, cyano, —COOH or Y—R₉;
Y represents —O—, —NRₐ—, —NRₐC(O)—, —C(O)NRₐ—, —C(O)—, —C(O)O—, —OC(O)—, —NRₐC(O)O— or —O(CH₂CH₂O)ₙ— wherein n is 1, 2, 3 or 4, and Rₐ and R_{b} both represents hydrogen;
R₉ represents (C₁-C₃)alkyl, (C₂-C₃)olefinic group, (C₃-C₆)cyclic hydrocarbon group, heterocyclyl, (C₂-C₃)alkynyl, (C₁-C₃)alkyl-(C₃-C₆)cyclic hydrocarbon or (C₁-C₃)alkyl-heterocyclyl, wherein R₉ may optionally be substituted by one or more substituents represented by R₁₀;
R₁₀ represents fluoro, chloro, hydroxy, trifluoromethyl, amino, (C₁-C₃)alkyl, (C₁-C₃)alkoxy, (C₁-C₃)alkylamino or (C₁-C₃)alkoxycarbonyl;
and pharmaceutically acceptable salts solvates or hydrates thereof.

Still another preferred embodiment of the present invention relates to compounds of formula I wherein R₁ is methyl; R₂ is 2-chloro; R₃ is 2-methyl and 4-fluoro, or 2-methyl and 4-bromo; R₄ is hydrogen or 4-chloro;
R₅ and R₆ independently represent hydrogen or (C₁-C₄)alkyl;
R₇ represents (C₁-C₆)alkyl, (C₃-C₆)cyclic hydrocarbon group, (C₂-C₆)olefinic group, heterocyclyl, (C₂-C₆)alkynyl, (C₁-C₆)alkyl-heterocyclyl, (C₁-C₆)alkyl-(C₃-C₆)cyclic hydrocarbon group, (C₂-C₆)olefinic group-heterocyclyl, (C₂-C₆), olefinic group-(C₃-C₆)cyclic hydrocarbon group, (C₂-C₆)alkynyl-heterocyclyl, (C₂-C₆)alkynyl-(C₃-C₆)cyclic hydrocarbon group; and wherein R₇ may optionally be substituted by one or more substituents represented by R₈;
R₈ represents halogen, hydroxy, trifluoromethyl, amino, (C₁-C₆)alkyl, (C₁-C₆)alkoxy, (C₁-C₆)alkylamino, (C₁-C₆)alkoxycarbonyl, (C₁-C₉)trialkylammonium in association with a pharmaceutically acceptable anion, cyano, —COOH or Y—R₉;
Y represents —O—, —NRₐ—, —NRₐC(O)—, —C(O)NRₐ—, —C(O)—, —C(O)O—, —OC(O)—, —NRₐC(O)O— or —O(CH₂CH₂O)ₙ— wherein n is 1, 2, 3 or 4, and Rₐ and R_{b} both represents hydrogen;
R₉ represents (C₁-C₃)alkyl, (C₂-C₃)olefinic group, (C₃-C₆)cyclic hydrocarbon group, heterocyclyl, (C₂-C₃)alkynyl, (C₁-C₃)alkyl-(C₃-C₆)cyclic hydrocarbon or (C₁-C₃)alkyl-heterocyclyl, wherein R₉ may optionally be substituted by one or more substituents represented by R₁₀;
R₁₀ represents fluoro, chloro, hydroxy, trifluoromethyl, amino, (C₁-C₃)alkyl, (C₁-C₃)alkoxy, (C₁-C₃)alkylamino or (C₁-C₃)alkoxycarbonyl;
and pharmaceutically acceptable salts solvates or hydrates thereof.

Yet another preferred embodiment of the present invention relates to compounds of formula I wherein R₁ is methyl; R₂ is 2-chloro; R is 2-methyl and 4-fluoro, or 2-methyl and 4-bromo; R₄ is hydrogen or 4-chloro;
R₅ and R₆ independently represent hydrogen or methyl;
R₇ represents methyl, ethyl, propyl, iso-propyl, butyl, tert-butyl, pentyl, heptyl, nonyl, 2-methyl-propyl, 1-methyl-propyl, 2,2-dimethyl-propyl, cyclopropyl, cyclobutyl, phenyl, ethenyl, propenyl, phenylmethyl, phenyl-1-allyl or 2-, 3- or 4-pyridyl, all of which may be substituted by R₈;
R₈ represents hydroxyl, carboxy;
Y represents —C(O)—O—, NH—C(O)—O, —O—, —O—C(O)— or —O(CH₂—CH₂—O)ₙ—, n being 3;
R₉ represents methyl, ethyl, tert-butyl or phenylmethyl;
R₁₀ represents fluoro, chloro, hydroxy, trifluoromethyl, amino, (C₁-C₃)alkyl, (C₁-C₃)alkoxy, (C₁-C₃)alkylamino or (C₁-C₃)alkoxycarbonyl;
and pharmaceutically acceptable salts, solvates and hydrates thereof.

Specific examples of compounds of formula I include
Succinic acid benzyl ester 1-[[3-chloro-4-(2-methyl-benzoyl)-phenyl]-(4-fluoro-2-methyl-phenyl)-carbamoyloxy]-ethyl ester;
Succinic acid mono-{1-[[3-chloro-4-(2-methyl-benzoyl)-phenyl]-(4-fluoro-2-methyl-phenyl)-carbamoyloxy]-ethyl}ester;
Sodium 3-{1-[[3-chloro-4-(2-methyl-benzoyl)-phenyl]-(4-fluoro-2-methyl-phenyl)-carbamoyloxy]-ethoxycarbonyl}-propionate;
{2-[2-(2-Methoxy-ethoxy)-ethoxy]-ethoxy}-acetic acid 1-[[3-chloro-4-(2-methyl-benzoyl)-phenyl]-(4-fluoro-2-methyl-phenyl)-carbamoyloxy]-ethyl ester;
{2-[2-(2-Methoxy-ethoxy)-ethoxy]-ethoxy}-acetic acid 1-{(4-bromo-2-methyl-phenyl)-[3-chloro-4-(2-methyl-benzoyl)-phenyl]-carbamoyloxy}-ethyl ester;
Succinic acid benzyl ester 1-{(4-bromo-2-methyl-phenyl)-[3-chloro-4-(2-methyl-benzoyl)-phenyl]-carbamoyloxy}-ethyl ester;
Succinic acid mono-(1-{(4-bromo-2-methyl-phenyl)-[3-chloro-4-(2-methyl-benzoyl)-phenyl]-carbamoyloxy}-ethyl) ester;
Succinic acid {(4-bromo-2-methyl-phenyl)-[3-chloro-4-(2-methyl-benzoyl)-phenyl]-carbamoyloxy}-methyl ester methyl ester;
Succinic acid benzyl ester {(4-bromo-2-methyl-phenyl)-[3-chloro-4-(2-methyl-benzoyl)-phenyl]-carbamoyloxy}-methyl ester;
Acetic acid 1-[[3-chloro-4-(2-methyl-benzoyl)-phenyl]-(4-fluoro-2-methyl-phenyl)-carbamoyloxy]-ethyl ester;
Propionic acid 1-[[3-chloro-4-(2-methyl-benzoyl)-phenyl]-(4-fluoro-2-methyl-phenyl)-carbamoyloxy]-ethyl ester;
Butyric acid 1-[[3-chloro-4-(2-methyl-benzoyl)-phenyl]-(4-fluoro-2-methyl-phenyl)-carbamoyloxy]-ethyl ester;
Butyric acid [[3-chloro-4-(2-methyl-benzoyl)-phenyl]-(4-fluoro-2-methyl-phenyl)-carbamoyloxy]-methyl ester;
Pentanoic acid 1-[[3-chloro-4-(2-methyl-benzoyl)-phenyl]-(4-fluoro-2-methyl-phenyl)-carbamoyloxy]-ethyl ester;
Hexanoic acid 1-[[3-chloro-4-(2-methyl-benzoyl)-phenyl]-(4-fluoro-2-methyl-phenyl)-carbamoyloxy]-ethyl ester;
Octanoic acid 1-[[3-chloro-4-(2-methyl-benzoyl)-phenyl]-(4-fluoro-2-methyl-phenyl)-carbamoyloxy]-ethyl ester;
Decanoic acid 1-[[3-chloro-4-(2-methyl-benzoyl)-phenyl]-(4-fluoro-2-methyl-phenyl)-carbamoyloxy]-ethyl ester;
Succinic acid 1-[[3-chloro-4-(2-methyl-benzoyl)-phenyl]-(4-fluoro-2-methyl-phenyl)-carbamoyloxy]-ethyl ester ethyl ester;
Methoxy-acetic acid 1-[[3-chloro-4-(2-methyl-benzoyl)-phenyl]-(4-fluoro-2-methyl-phenyl)-carbamoyloxy]-ethyl ester;
Methoxy-acetic acid [[3-chloro-4-(2-methyl-benzoyl)-phenyl]-(4-fluoro-2-methyl-phenyl)-carbamoyloxy]-methyl ester;
Butyric acid 1-[[3-chloro-4-(4-chloro-2-methyl-benzoyl)-phenyl]-(4-fluoro-2-methyl-phenyl)-carbamoyloxy]-ethyl ester;
3-Methoxy-propionic acid 1-[[3-chloro-4-(4-chloro-2-methyl-benzoyl)-phenyl]-(4-fluoro-2-methyl-phenyl)-carbamoyloxy]-ethyl ester;
3,3-Dimethyl-butyric acid [[3-chloro-4-(2-methyl-benzoyl)-phenyl]-(4-fluoro-2-methyl-phenyl)-carbamoyloxy]-methyl ester;
Cyclopropanecarboxylic acid [[3-chloro-4-(2-methyl-benzoyl)-phenyl]-(4-fluoro-2-methyl-phenyl)-carbamoyloxy]-methyl ester;
Cyclobutanecarboxylic acid [[3-chloro-4-(2-methyl-benzoyl)-phenyl]-(4-fluoro-2-methyl-phenyl)-carbamoyloxy]-methyl ester;
2-Hydroxy-propionic acid 1-[[3-chloro-4-(2-methyl-benzoyl)-phenyl]-(4-fluoro-2-methyl-phenyl)-carbamoyloxy]-ethyl ester;
2-Methyl-but-2-enoic acid 1-[[3-chloro-4-(2-methyl-benzoyl)-phenyl]-(4-fluoro-2-methyl-phenyl)-carbamoyloxy]-ethyl ester;
2-Hydroxy-2-methyl-propionic acid 1-[[3-chloro-4-(2-methyl-benzoyl)-phenyl]-(4-fluoro-2-methyl-phenyl)-carbamoyloxy]-ethyl ester;
2-Hydroxy-2-methyl-propionic acid 1-[[3-chloro-4-(4-chloro-2-methyl-benzoyl)-phenyl]-(4-fluoro-2-methyl-phenyl)-carbamoyloxy]-ethyl ester;
Isobutyric acid 1-[[3-chloro-4-(2-methyl-benzoyl)-phenyl]-(4-fluoro-2-methyl-phenyl)-carbamoyloxy]-ethyl ester;
Isobutyric acid [[3-chloro-4-(2-methyl-benzoyl)-phenyl]-(4-fluoro-2-methyl-phenyl)-carbamoyloxy]-methyl ester;
2,2-Dimethyl-propionic acid 1-[[3-chloro-4-(2-methyl-benzoyl)-phenyl]-(4-fluoro-2-methyl-phenyl)-carbamoyloxy]-ethyl ester;
3-Methyl-butyric acid 1-[[3-chloro-4-(2-methyl-benzoyl)-phenyl]-(4-fluoro-2-methyl-phenyl)-carbamoyloxy]-ethyl ester;
2-Methyl-butyric acid 1-[[3-chloro-4-(2-methyl-benzoyl)-phenyl]-(4-fluoro-2-methyl-phenyl)-carbamoyloxy]-ethyl ester;
Cyclopropanecarboxylic acid 1-[[3-chloro-4-(2-methyl-benzoyl)-phenyl]-(4-fluoro-2-methyl-phenyl)-carbamoyloxy]-ethyl ester;
Acrylic acid 1-[[3-chloro-4-(2-methyl-benzoyl)-phenyl]-(4-fluoro-2-methyl-phenyl)-carbamoyloxy]-ethyl ester;
But-2-enoic acid 1-[[3-chloro-4-(2-methyl-benzoyl)-phenyl]-(4-fluoro-2-methyl-phenyl)-carbamoyloxy]-ethyl ester;
But-2-enoic acid [[3-chloro-4-(2-methyl-benzoyl)-phenyl]-(4-fluoro-2-methyl-phenyl)-carbamoyloxy]-methyl ester;
Cyclobutanecarboxylic acid 1-[[3-chloro-4-(2-methyl-benzoyl)-phenyl]-(4-fluoro-2-methyl-phenyl)-carbamoyloxy]-ethyl ester;
3-Methoxy-propionic acid 1-[[3-chloro-4-(2-methyl-benzoyl)-phenyl]-(4-fluoro-2-methyl-phenyl)-carbamoyloxy]-ethyl ester;
2-Acetoxy-propionic acid 1-[[3-chloro-4-(2-methyl-benzoyl)-phenyl]-(4-fluoro-2-methyl-phenyl)-carbamoyloxy]-ethyl ester;
2,2-Dimethyl-propionic acid [[3-chloro-4-(2-methyl-benzoyl)-phenyl]-(4-fluoro-2-methyl-phenyl)-carbamoyloxy]-methyl ester;
3-Phenyl-acrylic acid 1-[[3-chloro-4-(2-methyl-benzoyl)-phenyl]-(4-fluoro-2-methyl-phenyl)-carbamoyloxy]-ethyl ester;
Benzoic acid 1-[[3-chloro-4-(2-methyl-benzoyl)-phenyl]-(4-fluoro-2-methyl-phenyl)-carbamoyloxy]-ethyl ester;
Pyridine-2-carboxylic acid 1-[[3-chloro-4-(2-methyl-benzoyl)-phenyl]-(4-fluoro-2-methyl-phenyl)-carbamoyloxy]-ethyl ester;
Isonicotinic acid 1-[[3-chloro-4-(2-methyl-benzoyl)-phenyl]-(4-fluoro-2-methyl-phenyl)-carbamoyloxy]-ethyl ester;
Nicotinic acid 1-[[3-chloro-4-(2-methyl-benzoyl)-phenyl]-(4-fluoro-2-methyl-phenyl)-carbamoyloxy]-ethyl ester;
Nicotinic acid 1-[[3-chloro-4-(4-chloro-2-methyl-benzoyl)-phenyl]-(4-fluoro-2-methyl-phenyl)-carbamoyloxy]-ethyl ester;
2-Hydroxy-benzoic acid 1-[[3-chloro-4-(2-methyl-benzoyl)-phenyl]-(4-fluoro-2-methyl-phenyl)-carbamoyloxy]-ethyl ester;
Hydroxy-phenyl-acetic acid 1-[[3-chloro-4-(2-methyl-benzoyl)-phenyl]-(4-fluoro-2-methyl-phenyl)-carbamoyloxy]-ethyl ester;
(S)-2-tert-Butoxycarbonylamino-3-hydroxy-propionic acid 1-[[3-chloro-4-(2-methyl-benzoyl)-phenyl]-(4-fluoro-2-methyl-phenyl)-carbamoyloxy]-ethyl ester (diastereomer A);
(S)-2-tert-Butoxycarbonylamino-3-hydroxy-propionic acid 1-[[3-chloro-4-(2-methyl-benzoyl)-phenyl]-(4-fluoro-2-methyl-phenyl)-carbamoyloxy]-ethyl ester (diastereomer B).

The compounds of the present invention are prodrugs of compounds known to be potent inhibitors of cytokines, such as IL-1β and TNF-α, possible due to an inhibition of P38 MAP kinase. The compounds of the present invention are therefore believed to be useful in the treatment of inflammatory diseases or states. In particular, the compounds may be useful in the treatment amelioration or prevention of inflammatory diseases or states of the skin, such as acne, atopic dermatitis, contact dermatitis and psoriasis. The compounds are also, as prodrugs of known inhibitors of cytokines, believed to be useful in the treatment, amelioration or prevention of systemic inflammatory diseases or states, such as asthma, allergy, arthritis, rheumatoid arthritis, spondyloarthritis, gout, atherosclerosis, chronic inflammatory bowel disease, uveitis and septic shock. The present invention therefore provides a method of treating, ameliorating or preventing acne, atopic dermatitis, psoriasis, asthma, allergy, arthritis, rheumatoid arthritis, spondyloarthritis, gout, atherosclerosis, chronic inflammatory bowel disease, uveitis and septic shock, the method comprising administering to a patient in need thereof an effective amount of a compound of formula I, optionally in combination with other therapeutically active compounds.

The prodrug moiety may endow the compounds of the present invention with particular advantages when used for the treatment of acne. The balanced hydrophilicity/hydrophobicity of said moiety may help targeting the compounds to the hydrophobic environment of the comedones. For other types of therapeutic interventions involving compounds of the present invention, e.g. systemic administration, the prodrug moiety will help the compounds to achieve a proper solubility to optimise the bioavailability.

A patient is an animal, including mammalians, and particularly humans. Animals also include domestic animals, such as horses, cows, sheep, swine, poultry, fish, cats, dogs, and zoo animals.

The term “effective amount” is intended to indicate an amount which gives rise to a therapeutic effect. The amount will vary, e.g. according to the age, size and sex of the patient, the disease and the severity of said disease, and the effect which is desired to achieve. It lies within the capabilities of any skilled physician or veterinary to determine what an effective amount is in any given situation. Suitable examples of “effective amounts” are the administration of 0.1-200 mg/kg body weight, such as 0.5-50 mg/kg body weight one or more times daily.

In therapeutic interventions comprising administration of compounds of the present invention, other therapeutically active compounds normally used in the treatment of the above indicated diseases may be used too. Such other therapeutically active compounds include glucocorticoids, vitamin D analogues, anti-histamines, platelet activating factor (PAF) antagonists, anticolinergic agents, methyl xanthines, β-adrenergic agents, COX-2 inhibitors, salicylates, indomethacin, flufenamate, naproxen, timegadine, gold salts, penicillamine, serum cholesterol-reducing agents, retinoids, zinc salts, and salicylazosulfapyridin (Salazopyrin). Administration of said therapeutically active compound may be concomitantly or sequentially to the administration of a compound of the present invention.

For use in therapy, compounds of the present invention may beneficially be presented in a pharmaceutical formulation. In a further aspect, the invention relates to a pharmaceutical composition comprising a compound of formula I, optionally together with another therapeutically active compound, and one or more pharmaceutically acceptable carriers or excipients. The carriers or excipients should be “pharmaceutically acceptable” in the sense of being compatible with the other ingredients of the formulations and not deleterious to the recipient thereof.

Conveniently, the active ingredient comprises from 0.1-100% by weight of the formulation. Conveniently, unit dose of a formulation contain between 50 mg and 5000 mg, preferably between 200 mg and 1000 mg of a compound of formula I.

By the term “unit dose” is meant a unitary, i.e. a single dose which is capable of being administered to a patient, and which may be readily handled and packed, remaining as a physically and chemically stable unit dose comprising either the active material as such or a mixture of it with solid or liquid pharmaceutical diluents or carriers.

The formulations include e.g. those in a form suitable for oral (including sustained or timed release), rectal, parenteral (including subcutaneous, intraperitoneal, intramuscular, intraarticular and intravenous), transdermal, ophthalmic, topical, nasal or buccal administration.

The formulations may conveniently be presented in unit dose form and may be prepared by any of the methods well known in the art of pharmacy, e.g. as disclosed in Remington, The Science and Practice of Pharmacy, 20^{th} ed., 2000. All methods include the step of bringing the active ingredient into association with the carrier, which constitutes one or more accessory ingredients. In general, the formulations are prepared by uniformly and intimately bringing the active ingredient into association with a liquid carrier or a finely divided solid carrier or both, and then, if necessary, shaping the product into the desired formulation.

Formulations of the present invention suitable for oral administration may be in the form of discrete units as capsules, sachets, tablets or lozenges, each containing a predetermined amount of the active ingredient; in the form of a powder or granules; in the form of a solution or a suspension in an aqueous liquid or non-aqueous liquid, such as ethanol or glycerol; or in the form of an oil-in-water emulsion or a water-in-oil emulsion. Such oils may be vegetable oils, such as e.g. cottonseed oil, sesame oil, coconut oil or peanut oil. Suitable dispersing or suspending agents for aqueous suspensions include synthetic or natural gums such as tragacanth, alginate, acacia, dextran, sodium carboxymethylcellulose, gelatin, methylcellulose, hydroxypropylmethylcellulose, hydroxypropylcellulose, carbomers and polyvinylpyrrolidone. The active ingredients may also be administered in the form of a bolus, electuary or paste.

A tablet may be made by-compressing or moulding the active ingredient optionally with one or more accessory ingredients. Compressed tablets may be prepared by compressing, in a suitable machine, the active ingredient(s) in a free-flowing form such as a powder or granules, optionally mixed by a binder, such as e.g. lactose, glucose, starch, gelatine, acacia gum, tragacanth gum, sodium alginate, carboxymethylcellulose, methylcellulose, hydroxypropylmethylcellulose, polyethylene glycol, waxes or the like; a lubricant such as e.g. sodium oleate, sodium stearate, magnesium stearate, sodium benzoate, sodium acetate, sodium chloride or the like; a disintegrating agent such as e.g. starch, methylcellulose, agar, bentonite, croscarmellose sodium, sodium starch glycollate, crospovidone or the like or a dispersing agent, such as polysorbate 80. Moulded tablets may be made by moulding, in a suitable machine, a mixture of the powdered active ingredient and suitable carrier moistened with an inert liquid diluent.

Formulations for rectal administration may be in the form of suppositories in which the compound of the present invention is admixed with low melting water soluble or insoluble solids such as cocoa butter, hydrogenated vegetable oils, polyethylene glycol or fatty acids esters of polyethylene glycols, while elixirs may be prepared using myristyl palmitate.

Formulations suitable for parenteral administration conveniently comprise a sterile oily or aqueous preparation of the active ingredients, which is preferably isotonic with the blood of the recipient, e.g. isotonic saline, isotonic glucose solution or buffer solution. The formulation may be conveniently sterilised by for instance filtration through a bacteria retaining filter, addition of sterilising agent to the formulation, irradiation of the formulation or heating of the formulation. Liposomal formulations as disclosed in e.g. Encyclopedia of Pharmaceutical Technology, vol. 9, 1994, are also suitable for parenteral administration.

Alternatively, the compound of formula I may be presented as a sterile, solid preparation, e.g. a freeze-dried powder, which is readily dissolved in a sterile solvent immediately prior to use.

Transdermal formulations may be in the form of a plaster or a patch.

Formulations suitable ophthalmic administration may be in the form of a sterile aqueous preparation of the active ingredients, which may be in microcrystalline form, for example, in the form of an aqueous microcrystalline suspension. Liposomal formulations or biodegradable polymer systems e.g. as disclosed in Encyclopedia of Pharmaceutical Technology, vol. 2, 1989, may also be used to present the active ingredient for ophthalmic administration.

Formulations suitable for topical or ophthalmic administration include liquid or semi-liquid preparations such as liniments, lotions, gels, applicants, oil-in-water or water-in-oil emulsions such as creams, ointments or pastes; or solutions or suspensions such as drops. Particularly suited dermal formulations are disclosed in WO 02/45752, example 1, test formulations A-M, the teaching of which is incorporated by reference herein in its entirety.

Formulations suitable for nasal or buccal administration include powder, self-propelling and spray formulations, such as aerosols and atomisers. Such formulations are disclosed in greater detail in e.g. Modern Pharmaceutics, 2^{nd} ed., G. S. Banker and C. T. Rhodes (Eds.), page 427-432, Marcel Dekker, New York; Modern Pharmaceutics, 3^{th} ed., G. S. Banker and C. T. Rhodes (Eds.), page 618-619 and 718-721, Marcel Dekker, New York and Encyclopedia of Pharmaceutical Technology vol. 10, J Swarbrick and J. C. Boylan (Eds), page 191-221, Marcel Dekker, New York.

Active transport forms of the present invention may also be delivered by use of monoclonale antibodies as individual carriers to which the compound molecules are coupled.

In addition to the aforementioned ingredients, the formulations of a compound of formula I may include one or more additional ingredients such as diluents, buffers, flavouring agents, colourant, surface active agents, thickeners, preservatives, e.g. methyl hydroxybenzoate (including anti-oxidants), emulsifying agents and the like.

Said other therapeutically active compounds include glucocorticoids, vitamin D analogues, anti-histamines, platelet activating factor (PAF) antagonists, anticolinergic agents, methyl xanthines, β-adrenergic agents, COX-2 inhibitors, salicylates, indomethacin, flufenamate, naproxen, timegadine, gold salts, penicillamine, serum cholesterol-reducing agents, retinoids, zinc salts, and salicylazosulfapyridin (Salazopyrin)

In still another embodiment, the invention relates to the use of compounds of formula I in the preparation of medicaments for use in the treatment of acne, atopic dermatitis, contact dermatitis, psoriasis, asthma, allergy, arthritis, rheumatoid arthritis, spondyloarthritis, gout, atherosclerosis, chronic inflammatory bowel disease, uveitis and septic shock.

Discolouring of Compounds of the Present Invention

The discolouring of compounds of formula I was investigated in a comparison with known aminobenzophenones.

Compounds were dissolved in DMSO at 100 mM. The solutions were diluted 1:10 in the test vehicle (Ethanol:Labrasol:water 65:25:10) just prior to the experiment. 10 ul aliquots were placed in droplets on filter paper and allowed to dry for 15 minutes into spots. Then the colour of the spots was scored using the following scale:

| | | |
|---|---|---|
| Score | 0: | No colour |
| | 1: | Very faint colour |
| | 2: | Light colour |
| | 3: | Medium colour |
| | 4: | Strong colour |

The spots were illuminated in a sun-test cabinet (Heraeus Suntest CPS) set for outdoor illumination for 5 minutes. The scoring was repeated after illumination. The results are shown in Table 1.

TABLE 1
Colour scores

| | Colour score prior | Colour score after |
|---|---|---|
| Compound | to illumination | illumination |
| Reference a | 0 | 3 |
| Reference b | 1 | 3 |
| Reference c | 1 | 3 |
| Compound 112 | 0 | 0 |
| Compound 113 | 0 | 0 |
| Compound 133 | 0 | 0 |
| Compound 147 | 0 | 0 |
| Compound 119 | 0 | 0 |

Reference a: 2-chloro-4-(4-fluoro-2-methyl-phenylamino)-2′-methylbenzophenone, compound 116 in WO 01/42189.
Reference b: 4-(2-amino-4-bromo-phenylamino)-2-chloro-2′-methylbenzophenone, compound 101 in WO 01/05744
Reference c: 4-(2-aminophenylamino)-2-chloro-2′-methylaminobenzophenon, compound 106 in WO 98/32730.

The above results clearly show that compounds of formula I have a significantly decreased, or even totally absent, discolouration when exposed to light. This property makes the compounds particular useful as medicament for treatment of dermal diseases.

Biological Activity

Inhibition of Cytokine Production

To study the effect of the compound of the present invention in vitro the inhibition of the IL-1β and TNF-α secretion was measured using the following procedure:

Cytokine production was measured in the media from lipopolysaccharide (LPS) stimulated peripheral blood mononuclear cells. The mononuclear cells were isolated from human peripheral blood by Lymphoprep® (Nycomed, Norway) fractionation and suspended in RPMI 1640 (growth medium) with foetal calf serum (FCS, 2%), at a concentration of 5×105 cells/ml. The cells were incubated in 24-well tissue culture plates in 1 ml aliquots. Test compounds were dissolved in dimethylsulfoxide (DMSO, 10 mM) and were diluted with the medium. Compounds were added to the cells for 30 minutes, then LPS (1 mg/ml final concentration) was added. The plates were incubated for 18 hours, and the concentration of IL-1β and TNF-α in the medium was determined by enzyme-linked immunosorbent assays. The median inhibitory concentrations (IC₅₀) of the compounds were calculated. The results are shown in Table 2.

TABLE 2
Inhibition of cytokines production in vitro
by compounds of formula I

| | The median inhibition concentration (IC₅₀, nM) | |
|---|---|---|
| | IL-1β | TNF-α |
| Reference a | 32 | 7.9 |
| compound 112 | 25 | 12 |
| compound 113 | 50 | 10 |
| compound 133 | 79 | 20 |
| compound 137 | 7.9 | 5.0 |
| compound 128 | 10 | 7.9 |

Reference a: 2-chloro-4-(4-fluoro-2-methyl-phenylamino)-2′-methylbenzophenone, compound 116 in WO 01/42189.

These results show that compounds of the present invention are able to inhibit the production of IL-1β and TNF-α, and showing a pharmacological activity comparable to the reference compounds, thus making them potentially useful in the treatment of inflammatory diseases.

Rhino Mouse Model

The rhino mouse is an in vivo model for the study of hyperplastic and comedolytic potency of compounds used in the treatment of acne. The rhino mouse has follicles on the skin, the orifices of which are distended with horny material, and these structures resembles human comedones.

The model uses mouse of the strain RHJ/LeJ Rhino, hr^{rh}/hr^{rh}. The mice are treated topically on the back daily for 21 days with the test compound. Compounds are tested for their ability to reduce the number of comedones.

The number of comedones in the skin of the mouse is determined by histological examination. The percentage change in the number of comedones compared to an untreated control group is calculated. The compounds were applied at 45 mM dissolved in acetone. Table 3 gives the results

TABLE 3
Reduction in number of comedones

| | Reduction in number |
|---|---|
| Compound | of comedones |
| Compound 112 | −56% |
| Compound 113 | −72% |
| Compound 133 | −61% |

The above data clearly show that compound of the present invention are capable of reducing the number of comedones, and thus useful in the treatment of acne.

Methods of Preparation

The compounds of the present invention can be prepared in a number of ways well known to those skilled in the art of organic synthesis. The compounds of the present invention can be synthesised using the methods outlined below, together with methods known in the art of synthetic organic chemistry, or variations thereof as appreciated by those skilled in the art. Preferred methods include, but are not limited to, those described below.

The compounds of formula I may be prepared using the reactions and techniques described in this section. The reactions are performed in solvents that are appropriate with respect to the reagents and materials employed and that are suitable for the transformations being effected. Also, in the synthetic methods described below, it is to be understood that all proposed reaction conditions, including choice of solvent, reaction atmosphere, reaction temperature, duration of experiment and work-up procedures, are chosen to be conditions of standard for that reaction, which should be readily recognised by one skilled in the art. It is understood by one skilled in the art of organic synthesis that the functionality present on various portions of the educt molecule must be compatible with the reagents and reactions proposed. Not all compounds of formula I falling into a given class may be compatible with some of the reaction conditions required in some of the methods described. Such restrictions to the substituents which are compatible with the reaction conditions will be readily apparent to one skilled in the art and alternative methods can be used.

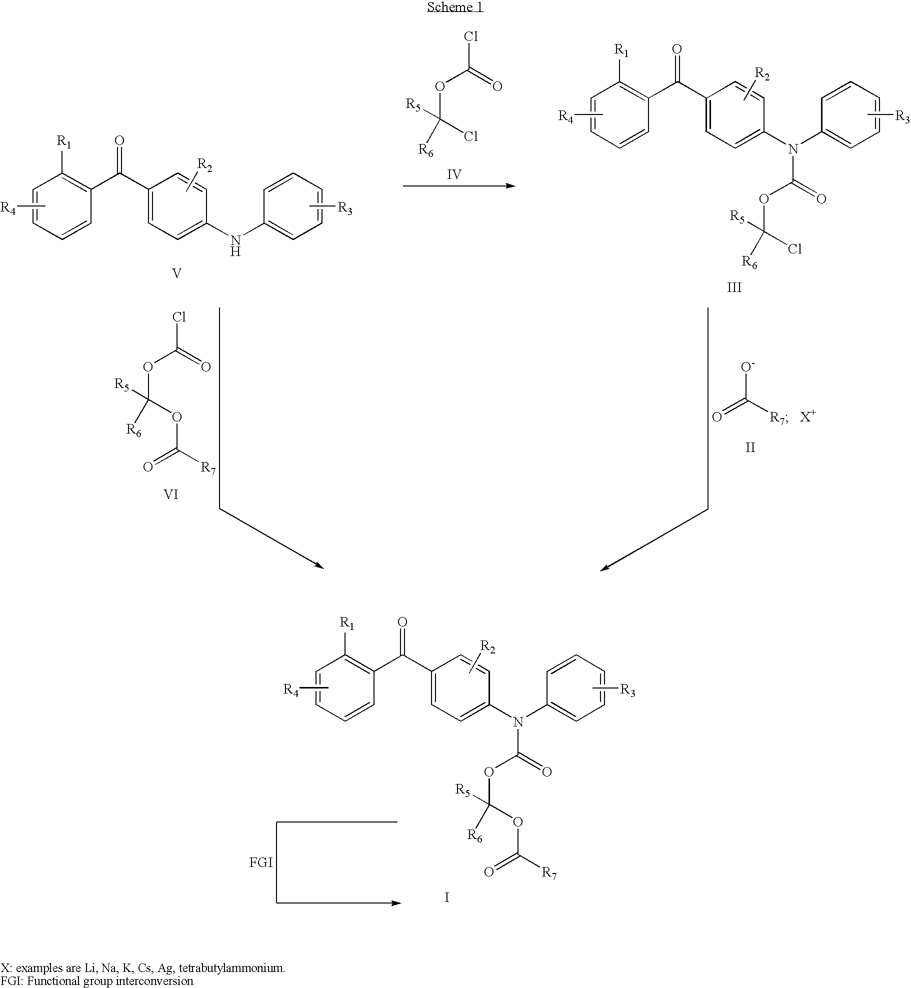

Compounds according to the present invention may be prepared by a process comprising coupling of a chloride of the formula III with a carboxylate of the formula II, as shown on scheme 1, or alternatively by a process comprising coupling of a diaraylamine of the formula V with a cabonchloridate with the formula VI, as shown on scheme 1, where R₁, R₂, R₃, R₄, R₅, R₆ and R₇ are as defined above, except that any substituents or functional groups which are potentially reactive in the coupling reactions may themselves be protected before the coupling reactions are performed and removed subsequently. The coupling reactions are typically performed at room temperature or below (−20 to 40 C.°) in an inert solvents like toluene, benzene, 1,4-dioxane, THF, diethyl ether and dichloromethane under an inert atmosphere, e.g argon or nitrogen.

Compounds according to the present invention may in special cases be prepared by a simple functional group interconversion (FGI), meaning a standard process, known to those skilled in the art of organic synthesis, where a functional group in compounds with the general formula I is transformed into a different functional group in one or more synthetic steps, leading to a new compound with the general formula I. Examples of such processes include, but are not limited to, hydrolysis of an ester to give an acid under basic conditions, deprotection of a tetrahydropyranylether to give an alcohol by treatment with e.g. a catalytic amount of acid, deprotection of a benzylic ester to give a carboxylic acid by catalytic hydrogenation and catalytic hydrogenation of an olefin to give a saturated hydrocarbon.

Compounds according to the general formulas IV and VI may be prepared as described in the literature (Folkmann, M., Lund, F. J.; Synthesis 1990, 1159), which is hereby incorporated as reference.

Compounds according to the general formula V may be prepared by the methods disclosed in WO 01/42189, which is hereby incorporated by reference in its entirety.

### EXAMPLES AND PREPARATIONS

The exemplified compounds are listed in Table 4.

All melting points are uncorrected. For ¹H nuclear magnetic resonance (NMR) spectra (300 MHz) and ¹³C NMR (75.6 MHz) chemical shift values (δ) (in ppm) are quoted, unless otherwise specified, for deuteriochloroform solutions relative to internal tetramethylsilane (δ=0.00) or chloroform (δ=7.25) or deuteriochloroform (δ=76.81 for ¹³C NMR) standard. The value of a multiplet, either defined (doublet (d), triplet (t), quartet (q)) or not (m) at the approximate mid point is given unless a range is quoted. The organic solvents used were anhydrous. Chromatography was performed on silica gel using the flash technique.

The following abbreviations have been used throughout:

| | |
|---|---|
| DCM | Dichloromethane |
| DMF | N,N-Dimethylformamide |
| MS | Mass spectroscopy |
| NMR | Nuclear magnetic resonance |
| RT | Room temperature |
| THF | Tetrahydrofuran |

The numbering in Table 4 refers to the numbering in the formula below

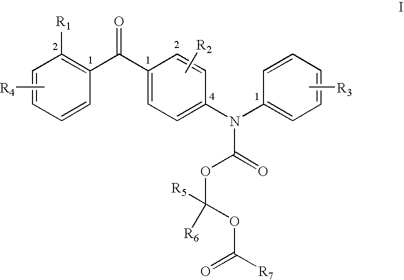

TABLE 4
Exemplified compounds with the general formula I.
(R₁ = methyl; R₂ = 2-Cl; R₃ = 2-CH₃,
4-F; R₄, and R₅ = H; unless otherwise noted).

| | Example | | | |
|---|---|---|---|---|
| Compound | no. | R₆ | R₇ | |
| 101 | 1 | —CH₃ | —CH₂CH₂COOBn | |
| 102 | 2 | —CH₃ | —CH₂CH₂COOH | |
| 103 | 3 | —CH₃ | —CH₂CH₂COONa | |
| 104 | 4 | —CH₃ | —CH₂(OCH₂CH₂)₃OCH₃ | |
| 105 | 5 | —CH₃ | —CH₂(OCH₂CH₂)₃OCH₃ | R₃ = 2-CH₃, 4-Br |
| 106 | 6 | —CH₃ | —CH₂CH₂COOBn | R₃ = 2-CH₃, 4-Br |
| 107 | 7 | —CH₃ | —CH₂CH₂COOH | R₃ = 2-CH₃, 4-Br |
| 108 | 8 | —H | —CH₂CH₂COOCH₃ | R₃ = 2-CH₃, 4-Br |
| 109 | 9 | —H | —CH₂CH₂COOBn | R₃ = 2-CH₃, 4-Br |
| 110 | 10 | —CH₃ | —CH₃ | |
| 111 | 11 | —CH₃ | —CH₂CH₃ | |
| 112 | 12 | —CH₃ | —CH₂CH₂CH₃ | |
| 113 | 13 | —H | —CH₂CH₂CH₃ | |
| 114 | 14 | —CH₃ | —CH₂CH₂CH₂CH₃ | |
| 115 | 15 | —CH₃ | —CH₂CH₂CH₂CH₂CH₃ | |
| 116 | 16 | —CH₃ | —CH₂CH₂CH₂CH₂CH₃CH₂CH₃ | |
| 117 | 17 | —CH₃ | —CH₂CH₂CH₂CH₂CH₂CH₂CH₃CH₂CH₃ | |
| 118 | 18 | —CH₃ | —CH₂CH₂COOCH₂CH₃ | |
| 119 | 19 | —CH₃ | —CH₂OCH₃ | |
| 120 | 20 | —H | —CH₂OCH₃ | |
| 121 | 21 | —CH₃ | —CH₂CH₂CH₃ | R₄ = 4-Cl |
| 122 | 22 | —CH₃ | —CH₂CH₂OCH₃ | R₄ = 4-Cl |
| 123 | 23 | —H | 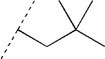 | |
| 124 | 24 | —H | 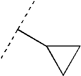 | |
| 125 | 25 | —H | 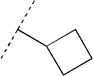 | |
| 126 | 26 | —CH₃ | 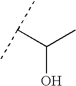 | |
| 127 | 27 | —CH₃ | 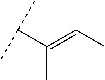 | |
| 128 | 28 | —CH₃ | 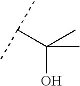 | |
| 129 | 29 | —CH₃ | 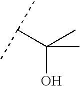 | R₄ = 4-Cl |
| 130 | 30 | —CH₃ | 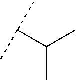 | |
| 131 | 31 | —H | 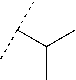 | |
| 132 | 32 | —CH₃ | 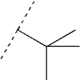 | |
| 133 | 33 | —CH₃ | 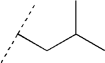 | |
| 134 | 34 | —CH₃ | 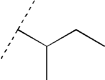 | |
| 135 | 35 | —CH₃ | 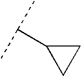 | |
| 136 | 36 | —CH₃ | 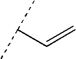 | |
| 137 | 37 | —CH₃ | 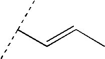 | |
| 138 | 38 | —H | 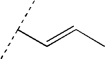 | |
| 139 | 39 | —CH₃ | 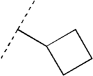 | |
| 140 | 40 | —CH₃ | 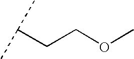 | |
| 141 | 41 | —CH₃ | 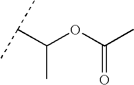 | |
| 142 | 42 | —H | 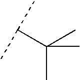 | |
| 143 | 43 | —CH₃ | 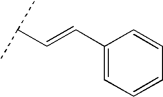 | |
| 144 | 44 | —CH₃ | 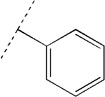 | |
| 145 | 45 | —CH₃ | 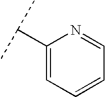 | |
| 146 | 46 | —CH₃ | 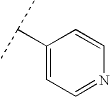 | |
| 147 | 47 | —CH₃ | 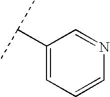 | |
| 148 | 48 | —CH₃ | 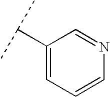 | R₄ = 4-Cl |
| 149 | 49 | —CH₃ | 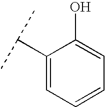 | |
| 150 | 50 | —CH₃ | 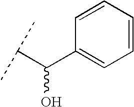 | |
| 151 | 51 | —CH₃ | 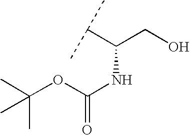 | diastereomer A |
| 152 | 52 | —CH₃ | 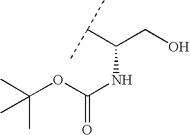 | diastereomer B |

### Preparation 1: [3-Chloro-4-(2-methyl-benzoyl)-phenyl]-(4-fluoro-2-methyl-phenyl)-carbamic acid 1-chloro-ethyl ester (compound 301)

The reaction was conducted under an atmosphere of argon. Sodium hydride (814 mg, 34 mmol) was added in small portions to a solution of [2-chloro-4-(4-fluoro-2-methyl-phenylamino)-phenyl]-o-tolyl-methanone (2.00 g, 5.65 mmol)( disclosed in WO 01/42189) in DMF (10 mL) at 0° C. under stirring. 1-Chloroethyl chloroformate (1.62 g, 11.3 mmol) was added and the reaction mixture was allowed to come to RT overnight. After 18 h at RT the mixture was poured into a mixture of saturated NH₄Cl (aq.) and EtOAc. The aqueous phase was extracted with more EtOAc (×2). The combined organic phases were washed with water, brine, dried (MgSO₄), filtered and concentrated in vacuo. The crude product was purified by flash chromatography using EtOAc/petroleum ether 1:8 as the eluent to afford the title compound as yellow oil.

### Preparation 2: (4-Bromo-2-methyl-phenyl)-[3-chloro-4-(2-methyl-benzoyl)-phenyl]-carbamic acid 1-chloro-ethyl ester (compound 302)

The reaction was conducted under an atmosphere of argon. Sodium hydride (1.04 g, 43 mmol) was added in small portions to a solution of [4-(4-bromo-2-methyl-phenylamino)-2-chloro-phenyl]-o-tolyl-methanone (3.0 g, 7.23 mmol)( disclosed in WO 01/42189) in DMF (25 mL) at 0° C. under stirring. 1-Chloroethyl chloroformate (2.07 g, 14.4 mmol) was added and the reaction mixture was allowed to come to RT overnight. After 18 h at RT the mixture was poured into a mixture of saturated NH₄Cl (aq.) and EtOAc. The aqueous phase was extracted with more EtOAc (×2). The combined organic phases were washed with water, brine, dried (MgSO₄), filtered and concentrated in vacuo to give the title compound. The crude product was used immediately without any further purification.

### Preparation 3: Succinic acid ethylsulfanylcarbonyloxymethyl ester methyl ester (compound 303)

A mixture of thiocarbonic acid S-ethyl ester O-iodomethyl ester (2.5 g, 10 mmol) (Synthesis 1990, 1159-1166) and potassium 3-methoxycarbonyl propionate (2.55 g, 15 mmol) in DMF (20 mL) was stirred overnight at RT. The reaction mixture was poured into a mixture of ice/water and diethyl ether. The aqueous phase was extracted with more diethyl ether. The combined organic phases were washed with 5% NaHCO₃, water, dried (MgSO₄), filtered and concentrated in vacuo. The crude product was purified by flash chromatography using diethyl ether/petroleum ether 1:1 as the eluent to afford the title compound as oil.

### Preparation 4: Succinic acid chlorocarbonyloxymethyl ester methyl ester (compound 304)

A solution of compound 303 (580 mg, 2.3 mmol) in redistilled sulphonylchloride (0.20 mL, 2.5 mmol) was stirred at 0° C. for 15 min and then at RT for 2 h. The reaction mixture was concentrated in vacuo and then co-evaporated with toluene (×2) to give the title compound as oil, which is only stable in solution (5.0 mL diethyl ether).

### Preparation 5: Succinic acid ethylsulfanylcarbonyloxymethyl ester benzyl ester (compound 305)

A solution of thiocarbonic acid S-ethyl ester O-iodomethyl ester (2.5 g, 10 mmol) (Synthesis 1990, 1159-1166) and silver 3-benzyloxycarbonyl propionate (3.5 g, 11 mmol) in DCM (1.00 mL) was stirred for 72 h at RT. The reaction mixture was filtered and washed with DCM and concentrated in vacuo. The crude product was purified by flash chromatography using diethyl ether/petroleum ether 1:1 as the eluent to afford the title compound as colourless oil.

### Preparation 6: Succinic acid chlorocarbonyloxymethyl ester benzyl ester (compound 306)

The reaction and work up was conducted as described in the preparation of compound 304. Starting compounds was compound 305 (1.00 g g, 3.0 mmol). The title compound was dissolved in THF (3.0 mL).

### Preparation 7: [3-Chloro-4-(2-methyl-benzoyl)-phenyl]-(4-fluoro-2-methyl-phenyl)-carbamic acid chloro-methyl ester (compound 307)

The reaction was conducted under an atmosphere of argon. A solution of potassium bis(trimethylsilyl)amide (34.77 mL, 0.5 M, 17.38 mmol) in toluene was added to a solution of [2-chloro-4-(4-fluoro-2-methyl-phenylamino)-phenyl]-o-tolyl-methanone (6.0 g, 17.0 mmol)( disclosed in WO 01/42189) in THF (170 mL) at −50° C. under stirring. After 15 min chloromethyl chloroformate (1.53 mL, 17.1 mmol) was added and the reaction mixture was stirred at −50° C. for 60 min and at RT for 60 min. The reaction mixture was washed with water, brine, dried (MgSO₄), filtered and concentrated in vacuo. The crude product was purified by flash chromatography using diethyl ether/petroleum ether 2:1 as the eluent to afford the title compound.

### Preparation 8: [3-Chloro-4-(4-chloro-2-methyl-benzoyl)-phenyl]-(4-fluoro-2-methyl-phenyl)-carbamic acid 1-chloro-ethyl ester (compound 308)

The reaction was conducted under an atmosphere of argon. A solution of potassium bis(trimethylsilyl)amide (8.6 mL, 0.5 M, 4.3 mmol) in toluene was added to a solution of [2-chloro-4-(4-fluoro-2-methyl-phenylamino)-phenyl]-(4-chloro-2-methyl-phenyl)-methanone (1.55 g, 4.00 mmol)(prepared by the methods disclosed in WO 01/42189) in THF (40 mL) at −50° C. under stirring. After 15 min 1-chloroethyl chloroformate (0.5 mL, 4.6 mmol) was added and the reaction mixture was stirred at −50° C. for 60 min and at RT for 60 min. The reaction mixture was washed with water, brine, dried (MgSO₄), filtered and concentrated in vacuo. The crude product was purified by flash chromatography using diethyl ether/petroleum ether 2:1 as the eluent to afford the title compound.

### Example 1

### Succinic acid benzyl ester 1-[[3-chloro-4-(2-methyl-benzoyl)-phenyl]-(4-fluoro-2-methyl-phenyl)-carbamoyloxy]-ethyl ester (compound 101)

The reactions were conducted under an argon atmosphere. Sodium 3-benzyloxycarbonyl-propionate (695 mg, 3.02 mmol) and tetrabutylamonium hydrogensulphate (256 mg, 0.76 mmol) was added to a solution of compound 301 (1.39 g, 3.02 mmol)) in DMF (10 mL) at 0° C. under stirring. The reaction mixture was stirred for 20 days at 5° C. after which it was poured into a mixture of water and EtOAc. The aqueous phase was extracted with more EtOAc. The combined organic phases were washed with water, brine, dried (MgSO₄), filtered and concentrated in vacuo. The crude product was purified by flash chromatography using EtOAc/petroleum ether 1:8 followed by 1:4 as the eluent to afford the title compound as foam.

¹³C NMR (CDCl₃): δ 196.5, 171.7, 170.4, 162.1 (d), 151.9, 144.3, 139.2, 137.2, 135.7, 135.5, 134.9 (d), 132.7, 131.9, 131.8, 131.0, 130.7, 128.6, 128.3, 128.2, 125.5, 124.9, 121.3, 118.1 (d), 114.3 (d), 90.6, 66.6, 28.9, 28.8, 21.0, 19.5, 17.8

### Example 2

### Succinic acid mono-{1-[[3-chloro-4-(2-methyl-benzoyl)-phenyl]-(4-fluoro-2-methyl-phenyl)-carbamoyloxy]-ethyl}ester (compound 102)

A solution of compound 101 (637 mg, 1.01 mmol) in EtOAc (7.0 mL) was added Pd/C (84 mg, 10%) and then hydrogenated under an atmosphere of hydrogen (1 atm.). After 5 h the reaction mixture was filtered through Decalite. The crude product was purified by flash chromatography using acetic acid/Et₂O/petroleum ether 0.02:1:1 as the eluent to afford the title compound as white solid.

¹³C NMR (CDCl₃): δ 196.7, 177.5, 170.3, 162.1 (d), 151.9, 144.3, 139.2, 138.7, 137.2, 135.5, 134.9 (d), 132.6, 131.9, 131.8, 131.0, 130.7, 125.5, 124.9, 121.4, 118.1 (d), 114.3 (d), 90.7, 28.7, 28.5, 21.0, 19.5, 17.8

### Example 3

### Sodium 3-{1-[[3-chloro-4-(2-methyl-benzoyl)-phenyl]-(4-fluoro-2-methyl-phenyl)-carbamoyloxy]-ethoxycarbonyl}-propionate (compound 103)

A solution of compound 102 (220 mg, 0.40 mmol) in acetone (1.5 mL) was mixed with a solution of sodium hydroxide (0.40 mL, 1.0 M, Aq.) in acetone (5.0 mL). The resulting solution was concentrated in vacuo and dried for 4 h in a freeze-dryer to give the title compound as white solid.

¹³C NMR (CDCl₃): δ 196.5, 171.8, 162.1 (d), 152.1, 144.2, 139.2, 137.1, 135.6, 134.9 (d), 132.6, 131.9, 131.8, 131.0, 130.7, 125.6, 125.0, 121.6, 118.0 (d), 114.3 (d), 90.6, 30.0, 29.7, 21.0, 19.4, 17.8

### Example 4

### {2-[2-(2-methoxy-ethoxy)-ethoxy]-ethoxy}-acetic acid 1-[[3-chloro-4-(2-methyl-benzoyl)-phenyl]-(4-fluoro-2-methyl-phenyl)-carbamoyloxy]-ethyl ester (compound 104)

The reactions were conducted under an argon atmosphere. To a solution of {2-[2-(2-methoxy-ethoxy)-ethoxy]-ethoxy}-acetic acid (434 mg, 1.95 mmol) in acetone (2.0 mL) was added tetrabutylammonium hydroxide (1.3 ml, 40% in water, 1.95 mmol) under stirring. After 10 min the reaction mixture was concentrated in vacuo (oil pump). A solution of compound 301 (898 mg, 1.95 mmol) in dry DMF (6.0 mL) was added to the residue. The reaction mixture was stirred 14 days at 10° C. after which it was poured into a mixture of water and EtOAc. The aqueous phase was extracted with more EtOAc. The combined organic phases were washed with water, brine, dried (MgSO₄), filtered and concentrated in vacuo. The crude product was purified by flash chromatography using EtOAc/petroleum ether 2:1 as the eluent to afford the title compound as yellow oil.

¹³C NMR (CDCl₃): δ 196.5, 168.7, 162.1 (d), 151.8, 144.2, 139.2, 138.7, 137.2, 135.6, 134.8 (d), 132.7, 131.9, 131.8, 131.0, 130.7, 125.5, 124.8, 121.3, 118.1 (d), 114.3 (d), 90.7, 72.0, 71.0, 70.6, 70.5, 68.3, 59.0, 21.0, 19.6, 17.9

### Example 5

### {2-[2-(2-methoxy-ethoxy)-ethoxy]-ethoxy}-acetic acid 1-{(4-bromo-2-methyl-phenyl)-[3-chloro-4-(2-methyl-benzoyl)-phenyl]-carbamoyloxy}-ethyl ester (compound 105)

The reaction and work up was conducted as described in the preparation of compound 104. Starting compounds were compound 302 (1.26 g g, 2.41 mmol) and {2-[2-(2-methoxy-ethoxy)-ethoxy]-ethoxy}-acetic acid (536 mg, 2.41 mmol). The crude product was purified by flash chromatography using Et₂O/petroleum ether 1:2 as the eluent to afford the title compound.

¹³C NMR (CDCl₃): δ 196.5, 168.7, 151.5, 143.9, 139.3, 138.4, 138.0, 137.1, 135.8, 134.4, 132.7, 131.9, 131.8, 131.0, 130.7, 130.6, 125.5, 124.9, 122.4, 121.4, 90.7, 71.9, 71.0, 70.6, 70.5, 68.3, 59.0, 21.0, 19.6, 17.6

### Example 6

### Succinic acid benzyl ester 1-{(4-bromo-2-methyl-phenyl)-[3-chloro-4-(2-methyl-benzoyl)-phenyl]-carbamoyloxy}-ethyl ester (compound 106)

The reaction and work up was conducted as described in the preparation of compound 104. Starting compounds were compound 302 (2.51 g g, 4.82 mmol) and sodium 3-benzyloxycarbonyl-propionate (1.11 g, 4.82 mmol). The crude product was purified by flash chromatography using Et₂O/petroleum ether 1:4 as the eluent to afford the title compound as brown oil.

¹³C NMR (CDCl₃): δ 196.5, 171.7, 170.4, 151.7, 144.0, 139.3, 138.1, 137.2, 135.7, 135.6, 134.4, 132.7, 131.9, 131.8, 131.0, 130.7, 130.6, 128.6, 128.3, 128.2, 125.5, 125.0, 125.0, 122.4, 121.5, 90.6, 66.6, 28.9, 28.8, 21.0, 19.5, 17.6

### Example 7

### Succinic acid mono-(1-{(4-bromo-2-methyl-phenyl)-[3-chloro-4-(2-methyl-benzoyl)-phenyl]-carbamoyloxy}-ethyl) ester (compound 107)

The reaction and work up was conducted as described in the preparation of compound 102. Starting compound was compound 106 (2.51 g g, 4.82 mmol). The crude product was purified by flash chromatography using EtOAc/petroleum ether 1:1 followed by EtOAc as the eluent to afford the title compound.

### Example 8

### Succinic acid {(4-bromo-2-methyl-phenyl)-[3-chloro-4-(2-methyl-benzoyl)-phenyl]-carbamoyloxy}-methyl ester methyl ester (compound 108)

The reaction was conducted under an argon atmosphere. To a stirred solution of [4-(4-bromo-2-methyl-phenylamino)-2-chloro-phenyl]-o-tolyl-methanone (415 mg, 1.00 mmol)(disclosed in WO 01/42189) in THF (10 mL) at −50° C. was added potassium bis(trimethylsilyl)amide (2.0 mL, 0.5 M in toluene). After 15 min a solution of compound 304 (2.05 ml, 1 mmol) in diethyl ether was added and the solution was stirred at RT for 18 h. The reaction mixture was poured into a mixture of ice-water and EtOAc. The aqueous phase was extracted with more EtOAc. The combined organic phases were washed with water, brine, dried (MgSO₄), filtered and concentrated in vacuo. The crude product was purified by flash chromatography using EtOAc/petroleum ether 1:1 as the eluent to afford the title compound as foam.

¹H NMR (CDCl₃): δ 7.47 (m, 1H), 7.43-7.35 (m, 4H), 7.31 (dd, 1H), 7.29 (m, 1H), 7.22-7.16 (m, 2H), 7.06 (d, 1H), 5.80 (s, 2H), 3.69 (s, 3H), 2.71-2.61 (m, 4H), 2.53 (s, 3H), 2.16 (s, 3H)

### Example 9

### Succinic acid benzyl ester {(4-bromo-2-methyl-phenyl)-[3-chloro-4-(2-methyl-benzoyl)-phenyl]-carbamoyloxy}-methyl ester (compound 109)

The reaction and work up was conducted as described in the preparation of compound 108. Starting compounds were compounds [4-(4-bromo-2-methyl-phenylamino)-2-chloro-phenyl]-o-tolyl-methanone (830 mg, 2.00 mmol)(disclosed in WO 01/42189) and compound 306 (2.10 mL, 2.1 mmol) The crude product was purified by flash chromatography using EtOAc/petroleum ether 1:1 as the eluent to afford the title compound.

¹H NMR (CDCl₃): δ 7.46 (m, 1H), 7.43-7.25 (m, 11H), 7.21-7.14 (m, 2H), 7.04 (d, 1H), 5.78 (s, 2H), 5.12 (s, 2H), 2.69 (s, 4H), 2.52 (s, 3H), 2.14 (s, 3H)

### Example 10

### Acetic acid 1-[[3-chloro-4-(2-methyl-benzoyl)-phenyl]-(4-fluoro-2-methyl-phenyl)-carbamoyloxy]-ethyl ester (compound 110)

To a solution of compound 301 (460 mg, 1.0 mmol) in THF (10.0 mL) was added tetrabutylammonium acetate (1.0 g, 3.3 mmol) under stirring. The reaction mixture was stirred for 18 h at RT after which it was washed with water, brine, dried (MgSO₄), filtered and concentrated in vacuo. The crude product was purified by flash chromatography using diethyl ether/petroleum ether 1:6 as the eluent to afford the title compound.

¹H NMR (CDCl₃): δ 7.44-7.25 (m, 5H), 7.22-7.10 (m, 3H), 7.05-6.92 (m, 2H), 6.86 (q, 1H), 2.52 (s, 3H), 2.18 (bs, 3H), 2.05 (s, 3H), 1.42 (d, 3H)

### Example 11

### Propionic acid 1-[[3-chloro-4-(2-methyl-benzoyl)-phenyl]-(4-fluoro-2-methyl-phenyl)-carbamoyloxy]-ethyl ester (compound 111)

To a solution of compound 301 (920 mg, 2.0 mmol) in THF (10.0 mL) was added tetrabutylammonium propionate (1.25 g, 4.0 mmol) under stirring. The reaction mixture was stirred for 18 h at RT after which it was washed with water, brine, dried (MgSO₄), filtered and concentrated in vacuo. The crude product was purified by flash chromatography using diethyl ether/petroleum ether 1:2 as the eluent to afford the title compound.

¹³C NMR (CDCl₃): δ 196.6, 172.5, 162.1 (d), 151.9, 144.3, 139.2, 138.8, 137.2, 135.4, 134.9 (d), 132.7, 131.9, 131.8, 131.0, 130.7, 125.5, 124.8, 121.2, 118.0 (d), 114.2 (d), 90.4, 27.4, 21.0, 19.5, 17.8, 8.8

### Example 12

### Butyric acid 1-[[3-chloro-4-(2-methyl-benzoyl)-phenyl]-(4-fluoro-2-methyl-phenyl)-carbamoyloxy]-ethyl ester (compound 112)

The reaction, work up and purification was conducted as described in the preparation of compound 111. Starting compounds were compound 301 (920 mg, 2.0 mmol) and tetrabutylammonium butyrate (1.0 g, 3.0 mmol).

¹³C NMR (CDCl₃): δ 196.6, 171.7, 162.1 (d), 151.9, 144.3, 139.2, 138.7, 137.2, 135.4, 134.9 (d), 132.7, 131.9, 131.8, 131.0, 130.7, 125.5, 124.8, 121.2, 118.0 (d), 114.2 (d), 90.4, 35.9, 21.0, 19.6, 18.2, 17.8, 13.5

### Example 13

### Butyric acid [[3-chloro-4-(2-methyl-benzoyl)-phenyl]-(4-fluoro-2-methyl-phenyl)-carbamoyloxy]-methyl ester (compound 113)

The reaction, work up and purification was conducted as described in the preparation of compound 111. Starting compounds were compound 307 (450 mg, 1.0 mmol) and tetrabutylammonium butyrate (495 mg, 1.5 mmol).

¹³C NMR (CDCl₃): δ 196.5, 172.0, 162.2 (d), 152.5, 144.1, 139.3, 138.6 (d), 137.1, 135.7, 134.7 (d), 132.7, 131.9, 131.8, 131.0, 130.7, 130.4 (d), 125.5, 124.8, 121.2, 118.2 (d), 114.4 (d), 80.9, 35.8, 21.0, 18.1, 17.8, 13.5

### Example 14

### Pentanoic acid 1-[[3-chloro-4-(2-methyl-benzoyl)-phenyl]-(4-fluoro-2-methyl-phenyl)-carbamoyloxy]-ethyl ester (compound 114)

The reaction, work up and purification was conducted as described in the preparation of compound 111. Starting compounds were compound 301 (620 mg, 1.5 mmol) and tetrabutylammonium pentanoate (855 mg, 2.5 mmol).

¹³C NMR (CDCl₃): δ 196.6, 171.8, 162.1 (d), 151.9, 144.3, 139.2, 138.7 (d), 137.3, 135.4, 134.9 (d), 132.7, 131.9, 131.8, 131.0, 130.7, 125.5, 124.8, 121.2, 118.0 (d), 114.2 (d), 90.4, 33.8, 26.7, 22.1, 21.0, 19.6, 17.8, 13.7

### Example 15

### Hexanoic acid 1-[[3-chloro-4-(2-methyl-benzoyl)-phenyl]-(4-fluoro-2-methyl-phenyl)-carbamoyloxy]-ethyl ester (compound 115)

The reaction, work up and purification was conducted as described in the preparation of compound 111. Starting compounds were compound 301 (1.40 g, 3.0 mmol) and tetrabutylammonium hexanoate (1.60 g, 4.47 mmol).

¹H NMR (CDCl₃): δ 7.44-7.25 (m, 5H), 7.22-7.10 (m, 3H), 7.05-6.91 (m, 2H), 6.86 (q, 1H), 2.52 (s, 3H), 2.28 (t, 2H), 2.17 (bs, 3H), 1.59 (m, 2H), 1.43 (d, 3H), 1.37-1.20 (m, 4H), 0.89 (bt, 3H)

### Example 16

### Octanoic acid 1-[[3-chloro-4-(2-methyl-benzoyl)-phenyl]-(4-fluoro-2-methyl-phenyl)-carbamoyloxy]-ethyl ester (compound 116)

The reaction, work up and purification was conducted as described in the preparation of compound 111. Starting compounds were compound 301 (1.0 g, 2.2 mmol) and tetrabutylammonium octanoate (1.3 g, 3.4 mmol).

¹H NMR (CDCl₃): δ 7.44-7.25 (m, 5H), 7.22-7.10 (m, 3H), 7.05-6.91 (m, 2H), 6.86 (q, 1H), 2.52 (s, 3H), 2.28 (t, 2H), 2.17 (bs, 3H), 1.59 (m, 2H), 1.43 (d, 3H), 1.36-1.20 (m, 8H), 0.88 (bt, 3H)

### Example 17

### Decanoic acid 1-[[3-chloro-4-(2-methyl-benzoyl)-phenyl]-(4-fluoro-2-methyl-phenyl)-carbamoyloxy]-ethyl ester (compound 117)

The reaction, work up and purification was conducted as described in the preparation of compound 111. Starting compounds were compound 301 (1.40 g, 3.0 mmol) and tetrabutylammonium decanoate (1.9 g, 4.5 mmol).

¹H NMR (CDCl₃): δ 7.44-7.25 (m, 5H), 7.22-7.10 (m, 3H), 7.05-6.91 (m, 2H), 6.86 (q, 1H), 2.52 (s, 3H), 2.28 (t, 2H), 2.17 (bs, 3H), 1.58 (m, 2H), 1.43 (bd, 3H), 1.38-1.20 (m, 12H), 0.88 (bt, 3H)

### Example 18

### Succinic acid 1-[[3-chloro-4-(2-methyl-benzoyl)-phenyl]-(4-fluoro-2-methyl-phenyl)-carbamoyloxy]-ethyl ester ethyl ester (compound 118)

The reaction, work up and purification was conducted as described in the preparation of compound 111. Starting compounds were compound 301 (460 mg, 1.00 mmol) and tetrabutylammonium 3-ethoxycarbonyl-propionate (600 mg, 1.5 mmol).

¹H NMR (CDCl₃): δ 7.44-7.25 (m, 5H), 7.22-7.10 (m, 3H), 7.05-6.93 (m, 2H), 6.89 (q, 1H), 4.14 (q, 2H), 2.68-2.54 (m, 4H), 2.52 (s, 3H), 2.17 (bs, 3H), 1.43 (d, 3H), 1.25 (t, 3H)

### Example 19

### Methoxy-acetic acid 1-[[3-chloro-4-(2-methyl-benzoyl)-phenyl]-(4-fluoro-2-methyl-phenyl)-carbamoyloxy]-ethyl ester (compound 119)

The reaction, work up and purification was conducted as described in the preparation of compound 111. Starting compounds were compound 301 (920 mg, 2.0 mmol) and tetrabutylammonium 2-methoxy-acetate (1.0 g, 3.0 mmol).

¹³C NMR (CDCl₃): δ 196.5, 168.5, 162.1 (d), 151.8, 144.1, 139.3, 138.6, 137.1, 135.6, 134.8 (d), 132.7, 131.9, 131.8, 131.0, 130.7, 125.5, 124.9, 121.3, 118.1 (d), 114.3 (d), 90.7, 69.4, 59.4, 21.0, 19.5, 17.9

### Example 20

### Methoxy-acetic acid [[3-chloro-4-(2-methyl-benzoyl)-phenyl]-(4-fluoro-2-methyl-phenyl)-carbamoyloxy]-methyl ester

The reaction and work up was conducted as described in the preparation of compound 111. Starting compounds were compound 307 (1.44 mL, 1.39 M in THF, 2.0 mmol) and tetrabutylammonium 2-methoxy-acetate (995 mg, 3.0 mmol). The crude product was purified by flash chromatography using diethyl ether/petroleum ether as a gradient from 1:2 to 2:1 as the eluent to afford the title compound.

¹H NMR (CDCl₃): δ 7.44-7.25 (m, 5H), 7.23-7.10 (m, 3H), 7.06-6.93 (m, 2H), 5.85 (bs, 2H), 4.08 (s, 2H), 3.45 (s, 3H), 2.53 (s, 3H), 2.17 (s, 3H)

### Example 21

### Butyric acid 1-[[3-chloro-4-(4-chloro-2-methyl-benzoyl)-phenyl]-(4-fluoro-2-methyl-phenyl)-carbamoyloxy]-ethyl ester (compound 121)

The reaction, work up and purification was conducted as described in the preparation of compound 111. Starting compounds were compound 308 (495 mg, 1.0 mmol) and tetrabutylammonium butyrate (500 mg, 1.5 mmol).

¹³C NMR (CDCl₃): δ 195.5, 171.7, 162.1 (d), 151.9, 144.6, 141.3, 138.8, 137.9, 135.7, 135.0, 134.9 (d), 132.7, 132.3, 131.8, 130.6, 125.8, 124.7, 121.2, 118.1 (d), 114.3 (d), 90.4, 35.9, 20.9, 19.6, 18.2, 17.8, 13.5

### Example 22

### 3-Methoxy-propionic acid 1-[[3-chloro-4-(4-chloro-2-methyl-benzoyl)-phenyl]-(4-fluoro-2-methyl-phenyl)-carbamoyloxy]-ethyl ester (compound 122)

The reaction, work up and purification was conducted as described in the preparation of compound 111. Starting compounds were compound 308 (495 mg, 1.0 mmol) and tetrabutylammonium 3-methoxy-propionate (520 mg, 1.5 mmol).

¹³C NMR (CDCl₃): δ 195.5, 169.7, 162.1 (d), 151.9, 144.6, 141.3, 138.9, 137.9, 135.7, 135.0, 134.8 (d), 132.6, 132.3, 131.8, 130.6, 125.8, 124.7, 121.3, 118.1 (d), 114.3 (d), 90.5, 67.5, 58.8, 34.8, 20.9, 19.5, 17.8

### Example 23

### 3,3-Dimethyl-butyric acid [[3-chloro-4-(2-methyl-benzoyl)-phenyl]-(4-fluoro-2-methyl-phenyl)-carbamoyloxy]-methyl ester (compound 123)

The reaction and work up was conducted as described in the preparation of compound 111. Starting compounds were compound 307 (1.44 mL, 1.39 M in THF, 2.0 mmol) and tetrabutylammonium 2,2-dimethyl-propionate (1.12 g, 3.0 mmol). The crude product was purified by flash chromatography using diethyl ether/petroleum ether as a gradient from 5:95 to 90:10 as the eluent to afford the title compound.

¹³C NMR (CDCl₃): δ 196.5, 170.6, 162.2 (d), 152.5, 144.1, 139.3, 138.6 (d), 137.1, 135.6, 134.7 (d), 132.7, 131.9, 131.8, 131.0, 130.7, 130.5 (d), 125.5, 124.8, 121.2, 118.2 (d), 114.4 (d), 80.9, 47.4, 30.8, 29.5, 21.0, 17.8

### Example 24

### Cyclopropanecarboxylic acid [[3-chloro-4-(2-methyl-benzoyl)-phenyl]-(4-fluoro-2-methyl-phenyl)-carbamoyloxy]-methyl ester (compound 124)

The reaction, work up and purification was conducted as described in the preparation of compound 111. Starting compounds were compound 307 (531 mg, 1.19 mmol) and tetrabutylammonium cyclopropanecarboxylate (583 mg, 1.78 mmol).

¹³C NMR (CDCl₃): δ 196.5, 173.4, 162.2 (d), 152.6, 144.2, 139.3, 138.6 (d), 137.2, 135.7, 134.7 (d), 132.7, 131.9, 131.8, 131.0, 130.7, 130.5 (d), 125.5, 124.8, 121.2, 118.2 (d), 114.4 (d), 80.9, 21.0, 17.8, 12.6, 9.1

### Example 25

### Cyclobutanecarboxylic acid [[3-chloro-4-(2-methyl-benzoyl)-phenyl]-(4-fluoro-2-methyl-phenyl)-carbamoyloxy]-methyl ester (compound 125)

The reaction and work up was conducted as described in the preparation of compound 111. Starting compounds were compound 307 (1.44 mL, 1.39 M in THF, 2.0 mmol) and tetrabutylammonium cyclobutanecarboxylate (1.02 g, 3.0 mmol). The crude product was purified by flash chromatography using diethyl ether/petroleum ether as a gradient from 0:100 to 40:60 as the eluent to afford the title compound.

¹H NMR (CDCl₃): δ 7.44-7.25 (m, 5H), 7.23-7.10 (m, 3H), 7.06-6.93 (m, 2H), 5.78 (m, 2H), 3.17 (m, 1H), 2.53 (s, 3H), 2.37-2.10 (m, 4H), 2.16 (s, 3H), 1.98 (m, 2H)

### Example 26

### 2-Hydroxy-propionic acid 1-[[3-chloro-4-(2-methyl-benzoyl)-phenyl]-(4-fluoro-2-methyl-phenyl)-carbamoyloxy]-ethyl ester (compound 126)

The reaction and work up was conducted as described in the preparation of compound 111. Starting compounds were compound 301 (920 mg, 2.0 mmol) and tetrabutylammonium 2-hydroxy-propionate (1.0 g, 3.0 mmol). The crude product was purified by flash chromatography using diethyl ether/petroleum ether as a gradient from 1:2 to 2:1 as the eluent to afford the title compound.

¹³C NMR (CDCl₃): δ 196.5, 173.8, 162.2 (d), 151.8, 144.1, 139.3, 138.5, 137.1, 135.7, 134.8 (d), 132.7, 132.0, 131.8, 131.0, 130.7, 130.5, 125.5, 124.9, 121.3, 118.1 (d), 114.4 (d), 91.1, 66.6, 21.0, 20.1, 19.5, 17.8

### Example 27

### (E)-2-Methyl-but-2-enoic acid 1-[[3-chloro-4-(2-methyl-benzoyl)-phenyl]-(4-fluoro-2-methyl-phenyl)-carbamoyloxy]-ethyl ester (compound 127)

The reaction and work up was conducted as described in the preparation of compound 111 except that the reaction was stopped after 3 h. Starting compounds were compound 301 (920 mg, 2.0 mmol) and tetrabutylammonium (E)-2-methyl-but-2-enoate (1.03 g, 3.0 mmol). The crude product was purified by flash chromatography using diethyl ether/petroleum ether as a gradient from 1:2 to 2:1 as the eluent to afford the title compound.

¹³C NMR (CDCl₃): δ 196.6, 165.9, 162.1 (d), 152.0, 144.4, 139.2, 139.0, 138.7, 137.3, 135.4, 135.0 (d), 132.7, 131.9, 131.8, 131.0, 130.6, 127.8, 125.5, 124.9, 121.3, 118.0 (d), 114.2 (d), 90.7, 21.0, 19.7, 17.8, 14.5, 11.9

### Example 28

### 2-Hydroxy-2-methyl-propionic acid 1-[[3-chloro-4-(2-methyl-benzoyl)-phenyl]-(4-fluoro-2-methyl-phenyl)-carbamoyloxy]-ethyl ester (compound 128)

The reaction, work up and purification was conducted as described in the preparation of compound 111. Starting compounds were compound 301 (920 mg, 2.0 mmol) and tetrabutylammonium 2-hydroxy-2-methyl-propionate (1.2 g, 3.5 mmol).

¹³C NMR (CDCl₃): δ 196.5, 175.6, 162.1 (d), 151.7, 144.1, 139.3, 138.6 (d), 137.1, 135.6, 134.7 (d), 132.7, 132.0, 131.8, 131.0, 130.7, 125.5, 124.7, 121.2, 118.1 (d), 114.3 (d), 91.3, 71.9, 27.0, 26.8, 21.0, 19.4, 17.8

### Example 29

### 2-Hydroxy-2-methyl-propionic acid 1-[[3-chloro-4-(4-chloro-2-methyl-benzoyl)-phenyl]-(4-fluoro-2-methyl-phenyl)-carbamoyloxy]-ethyl ester (compound 129)

The reaction and work up was conducted as described in the preparation of compound 111. Starting compounds were compound 308 (495 mg, 1.0 mmol) and tetrabutylammonium 2-hydroxy-2-methyl-propionate (550 mg, 1.6 mmol). The crude product was purified by flash chromatography using diethyl ether/petroleum ether 1:1 as the eluent to afford the title compound.

¹³C NMR (CDCl₃): δ 195.4, 175.6, 162.2 (d), 151.7, 144.3, 141.3, 138.7, 138.0, 135.6, 135.2, 134.7, 132.7, 132.3, 131.8, 130.6, 130.5 (d), 125.8, 124.7, 121.2, 118.1 (d), 114.4 (d), 91.3, 71.9, 27.0, 26.8, 20.9, 19.4, 17.8

### Example 30

### Isobutyric acid 1-[[3-chloro-4-(2-methyl-benzoyl)-phenyl]-(4-fluoro-2-methyl-phenyl)-carbamoyloxy]-ethyl ester (compound 130)

The reaction, work up and purification was conducted as described in the preparation of compound 111. Starting compounds were compound 301 (460 mg, 1.00 mmol) and tetrabutylammonium 2-methyl-propionate (550 mg, 1.66 mmol).

¹³C NMR (CDCl₃): δ 196.6, 175.1, 162.1 (d), 151.9, 144.3, 139.2, 138.7, 137.2, 135.4, 135.0 (d), 132.7, 131.9, 131.8, 131.0, 130.7, 125.5, 124.7, 121.2, 118.0 (d), 114.2 (d), 90.4, 33.8, 21.0, 19.5, 18.8, 18.5, 17.8

### Example 31

### Isobutyric acid [[3-chloro-4-(2-methyl-benzoyl)-phenyl]-(4-fluoro-2-methyl-phenyl)-carbamoyloxy]-methyl ester (compound 131)

The reaction, work up and purification was conducted as described in the preparation of compound 111. Starting compounds were compound 307 (540 mg, 1.20 mmol) and tetrabutylammonium 2-methyl-propionate (760 mg, 2.30 mmol).

¹³C NMR (CDCl₃): δ 196.5, 175.5, 162.2 (d), 152.5, 144.2, 139.3, 138.6 (d), 137.2, 135.7, 134.7 (d), 132.7, 131.9, 131.8, 131.0, 130.7, 130.5 (d), 125.5, 124.8, 121.2, 118.2 (d), 114.4 (d), 81.0, 33.8, 21.0, 18.7, 17.8

### Example 32

### 2,2-Dimethyl-propionic acid 1-[[3-chloro-4-(2-methyl-benzoyl)-phenyl]-(4-fluoro-2-methyl-phenyl)-carbamoyloxy]-ethyl ester (compound 132)

The reaction, work up and purification was conducted as described in the preparation of compound 111. Starting compounds were compound 301 (460 mg, 1.00 mmol) and tetrabutylammonium 2,2-dimethyl-propionate (570 mg, 1.66 mmol).

¹³C NMR (CDCl₃): δ 196.6, 176.5, 162.1 (d), 151.9, 144.4, 139.2, 138.8, 137.3, 135.4, 135.0 (d), 132.7, 131.9, 131.8, 131.0, 130.7, 125.5, 124.7, 121.1, 118.0 (d), 114.2 (d), 90.6, 38.6, 26.8, 21.0, 19.4, 17.8

### Example 33

### 3-Methyl-butyric acid 1-[[3-chloro-4-(2-methyl-benzoyl)-phenyl]-(4-fluoro-2-methyl-phenyl)-carbamoyloxy]-ethyl ester (compound 133)

The reaction, work up and purification was conducted as described in the preparation of compound 111. Starting compounds were compound 301 (690 mg, 1.50 mmol) and tetrabutylammonium 3-methyl-butanoate (860 mg, 2.50 mmol).

¹H NMR (CDCl₃): δ 7.44-7.25 (m, 5H), 7.22-7.10 (m, 3H), 7.04-6.91 (m, 2H), 6.86 (q, 1H), 2.52 (s, 3H), 2.17 (bs, 3H), 2.16 (d, 2H), 2.07 (m, 1H), 1.44 (d, 3H), 0.92 (d, 6H)

### Example 34

### 2-Methyl-butyric acid 1-[[3-chloro-4-(2-methyl-benzoyl)-phenyl]-(4-fluoro-2-methyl-phenyl)-carbamoyloxy]-ethyl ester (compound 134)

The reaction, work up and purification was conducted as described in the preparation of compound 111. Starting compounds were compound 301 (460 mg, 1.00 mmol) and tetrabutylammonium 2-methyl-butanoate (570 mg, 1.66 mmol).

¹H NMR (CDCl₃): δ 7.44-7.25 (m, 5H), 7.22-7.10 (m, 3H), 7.04-6.90 (m, 2H), 6.86 (q, 1H), 2.52 (s, 3H), 2.33 (m, 1H), 2.17 (bs, 3H), 1.74-1.34 (m, 2H), 1.44 (bd, 3H), 1.14-1.07 (d, 3H), 0.91-0.79 (t, 3H)

### Example 35

### Cyclopropanecarboxylic acid 1-[[3-chloro-4-(2-methyl-benzoyl)-phenyl]-(4-fluoro-2-methyl-phenyl)-carbamoyloxy]-ethyl ester (compound 135)

The reaction, work up and purification was conducted as described in the preparation of compound 111. Starting compounds were compound 301 (460 mg, 1.00 mmol) and tetrabutylammonium cyclopropanecarboxylate (500 mg, 1.50 mmol).

¹H NMR (CDCl₃): δ 7.44-7.25 (m, 5H), 7.22-7.10 (m, 3H), 7.05-6.92 (m, 2H), 6.86 (q, 1H), 2.52 (s, 3H), 2.17 (bs, 3H), 1.56 (m, 1H), 1.44 (bd, 3H), 1.01 (m, 2H), 0.90 (m, 2H)

### Example 36

### Acrylic acid 1-[[3-chloro-4-(2-methyl-benzoyl)-phenyl]-(4-fluoro-2 -methyl-phenyl)-carbamoyloxy]-ethyl ester (compound 136)

The reaction, work up and purification was conducted as described in the preparation of compound 111. Starting compounds were compound 301 (460 mg, 1.00 mmol) and tetrabutylammonium acrylate (520 mg, 1.66 mmol).

¹H NMR (CDCl₃): δ 7.44-7.25 (m, 5H), 7.22-7.10 (m, 3H), 7.05-6.89 (m, 3H), 6.46 (dd, 1H, J=17.2 Hz and 1.5 Hz), 6.08 (dd, 1H, J=17.2 Hz and 10.3 Hz), 5.90 (dd, 1H, J=10.3 Hz and 1.5 Hz), 2.52 (s, 3H), 2.17 (bs, 3H), 1.47 (bd, 3H)

### Example 37

### (E)-But-2-enoic acid 1-[[3-chloro-4-(2-methyl-benzoyl)-phenyl]-(4-fluoro-2-methyl-phenyl)-carbamoyloxy]-ethyl ester (compound 137)

The reaction, work up and purification was conducted as described in the preparation of compound 111. Starting compounds were compound 301 (460 mg, 1.00 mmol) and tetrabutylammonium (E)-but-3-enoate (500 mg, 1.50 mmol).

¹H NMR (CDCl₃): δ 7.44-7.25 (m, 5H), 7.22-6.89 (m, 7H), 5.80 (dq, 1H, J=15.6 Hz and 1.6 Hz), 2.52 (s, 3H), 2.17 (bs, 3H), 1.89 (dd, 3H), 1.45 (bd, 3H)

### Example 38

### (E)-But-2-enoic acid [[3-chloro-4-(2-methyl-benzoyl)-phenyl]-(4-fluoro-2-methyl-phenyl)-carbamoyloxy]-methyl ester (compound 138)

The reaction, work up and purification was conducted as described in the preparation of compound 111. Starting compounds were compound 307 (446 mg, 1.00 mmol) and tetrabutylammonium (E)-but-3-enoate (491 mg, 1.5 mmol).

¹³C NMR (CDCl₃): δ 196.5, 164.6, 162.2 (d), 152.7, 147.5, 144.2, 139.3, 138.6 (d), 137.2, 135.7, 134.7 (d), 132.7, 131.9, 131.8, 131.0, 130.7, 130.5 (d), 125.5, 124.8, 121.4, 121.3, 118.2 (d), 114.4 (d), 80.8, 21.0, 18.2, 17.8

### Example 39

### Cyclobutanecarboxylic acid 1-[[3-chloro-4-(2-methyl-benzoyl)-phenyl]-(4-fluoro-2-methyl-phenyl)-carbamoyloxy]-ethyl ester (compound 139)

The reaction and work up was conducted as described in the preparation of compound 111. Starting compounds were compound 301 (920 mg, 2.00 mmol) and tetrabutylammonium cyclobutanecarboxylate (1.2 g, 3.5 mmol). The crude product was purified by flash chromatography using diethyl ether/petroleum ether 1:1 as the eluent to afford the title compound.

¹³C NMR (CDCl₃): δ 196.6, 173.5, 162.1 (d), 152.0, 144.4, 139.2, 138.8, 137.2, 135.4, 135.0 (d), 132.7, 131.9, 131.8, 131.0, 130.7, 125.5, 124.8, 121.2, 118.0 (d), 114.2 (d), 90.4, 37.7, 25.1, 24.8, 21.0, 19.5, 18.4, 17.8

### Example 40

### 3-Methoxy-propionic acid 1-[[3-chloro-4-(2-methyl-benzoyl)-phenyl]-(4-fluoro-2-methyl-phenyl)-carbamoyloxy]-ethyl ester (compound 140)

The reaction and work up was conducted as described in the preparation of compound 111. Starting compounds were compound 301 (920 mg, 2.0 mmol) and tetrabutylammonium 3-methoxy-propionate (1.04 g, 3.0 mmol). The crude product was purified by flash chromatography using diethyl ether/petroleum ether as a gradient from 1:9 to 3:2 as the eluent to afford the title compound.

¹³C NMR (CDCl₃): δ 196.6, 169.7, 162.1 (d), 151.9, 144.3, 139.2, 138.6, 137.2, 135.5, 134.9 (d), 132.7, 131.9, 131.8, 131.0, 130.7, 125.5, 124.8, 121.3, 118.1 (d), 114.3 (d), 90.5, 67.5, 58.8, 34.8, 21.0, 19.5, 17.8

### Example 41

### 2-Acetoxy-propionic acid 1-[[3-chloro-4-(2-methyl-benzoyl)-phenyl]-(4-fluoro-2-methyl-phenyl)-carbamoyloxy]-ethyl ester (compound 141)

The reaction and work up was conducted as described in the preparation of compound 111. Starting compounds were compound 301 (920 mg, 2.0 mmol) and tetrabutylammonium 2-acetoxy-propionate (1.12 g, 3.0 mmol). The crude product was purified by flash chromatography using diethyl ether/petroleum ether as a gradient from 1:9 to 1:1 as the eluent to afford the title compound.

¹³C NMR (CDCl₃): δ 196.5, 170.3, 169.0, 162.2 (d), 151.7, 144.2, 139.3, 137.2, 135.6, 134.8, 132.7, 132.3, 131.9, 131.8, 131.0, 130.7, 125.5, 124.8, 121.2, 118.1 (d), 114.3 (d), 91.1, 90.8, 68.2, 21.0, 20.6, 19.4, 17.8, 16.7, 16.6

### Example 42

### 2,2-Dimethyl-propionic acid [[3-chloro-4-(2-methyl-benzoyl)-phenyl]-(4-fluoro-2-methyl-phenyl)-carbamoyloxy]-methyl ester (compound 142)

The reaction, work up and purification was conducted as described in the preparation of compound 111. Starting compounds were compound 307 (0.72 mL, 1.39 M in THF, 1.00 mmol) and tetrabutylammonium 2,2-dimethyl-propionate (516 mg, 1.5 mmol).

¹³C NMR (CDCl₃): δ 196.5, 176.9, 162.2 (d), 152.4, 144.2, 139.3, 138.7 (d), 137.2, 135.7, 134.8 (d), 132.7, 131.9, 131.8, 131.0, 130.7, 130.5 (d), 125.5, 124.8, 121.2, 118.2 (d), 114.4 (d), 81.2, 38.8, 26.9, 21.0, 17.8

### Example 43

### 3-Phenyl-acrylic acid 1-[[3-chloro-4-(2-methyl-benzoyl)-phenyl]-(4-fluoro-2-methyl-phenyl)-carbamoyloxy]-ethyl ester (compound 143)

The reaction and work up was conducted as described in the preparation of compound 111 except that the reaction was stopped after 4 h. Starting compounds were compound 301 (920 mg, 2.0 mmol) and tetrabutylammonium 3-phenyl-acrylate (1.17 g, 3.0 mmol). The crude product was purified by flash chromatography using diethyl ether/petroleum ether as a gradient from 1:9 to 3:2 as the eluent to afford the title compound.

¹³C NMR (CDCl₃): δ 196.6, 164.8, 162.1 (d), 152.0, 146.5, 144.4, 139.2, 137.2, 135.5, 135.0 (d), 134.1, 132.7, 131.9, 131.8, 131.0, 130.7, 130.6, 129.0, 128.3, 125.5, 124.9, 121.3, 118.1 (d), 116.9, 114.3 (d), 90.6, 21.0, 19.7, 17.9

### Example 44

### Benzoic acid 1-[[3-chloro-4-(2-methyl-benzoyl)-phenyl]-(4-fluoro-2-methyl-phenyl)-carbamoyloxy]-ethyl ester (compound 144)

The reaction and work up was conducted as described in the preparation of compound 111 except that the reaction was stopped after 1.5 h. Starting compounds were compound 301 (920 mg, 2.0 mmol) and tetrabutylammonium benzoate (1.09 g, 3.0 mmol). The crude product was purified by flash chromatography using diethyl ether/petroleum ether as a gradient from 1:9 to 2:3 as the eluent to afford the title compound.

¹³C NMR (CDCl₃): δ 196.6, 164.6, 162.1 (d), 152.0, 144.3, 139.2, 138.7, 137.2, 135.5, 134.9 (d), 133.6, 132.7, 131.9, 131.8, 131.0, 130.6, 129.8, 129.2, 128.5, 125.5, 124.9, 121.3, 118.0 (d), 114.3 (d), 91.0, 21.0, 19.7, 17.8

### Example 45

### Pyridine-2-carboxylic acid 1-[[3-chloro-4-(2-methyl-benzoyl)-phenyl]-(4-fluoro-2-methyl-phenyl)-carbamoyloxy]-ethyl ester (compound 145)

The reaction and work up was conducted as described in the preparation of compound 111. Starting compounds were compound 301 (920 mg, 2.0 mmol) and tetrabutylammonium pyridine-2-carboxylate (1.09 g, 3.0 mmol). The crude product was purified by flash chromatography using diethyl ether/petroleum ether as a gradient from 1:2 to 1:2 as the eluent to afford the title compound.

¹³C NMR (CDCl₃): δ 196.5, 163.4, 162.1 (d), 152.0, 150.1, 147.2, 144.3, 139.2, 138.7, 137.2, 137.1, 135.5, 134.9 (d), 132.7, 131.9, 131.8, 131.0, 130.6, 127.3, 125.6, 125.5, 124.9, 121.4, 118.1 (d), 114.3 (d), 91.5, 21.0, 19.7, 17.9

### Example 46

### Isonicotinic acid 1-[[3-chloro-4-(2-methyl-benzoyl)-phenyl]-(4-fluoro-2-methyl-phenyl)-carbamoyloxy]-ethyl ester (compound 146)

The reaction and work up was conducted as described in the preparation of compound 111. Starting compounds were compound 301 (920 mg, 2.0 mmol) and tetrabutylammonium isonicotinate (1.09 g, 3.0 mmol). The crude product was purified by flash chromatography using diethyl ether/petroleum ether as a gradient from 1:2 to 1:2 as the eluent to afford the title compound.

¹³C NMR (CDCl₃): δ 196.5, 163.3, 162.1 (d), 151.8, 150.8, 144.1, 139.3, 138.6 (d), 137.1, 136.4, 135.7, 134.8 (d), 132.7, 131.9, 131.8, 131.0, 130.7, 130.5 (d), 125.5, 124.9, 122.9, 121.3, 118.1 (d), 114.4 (d), 91.4, 21.0, 19.6, 17.9

### Example 47

### Nicotinic acid 1-[[3-chloro-4-(2-methyl-benzoyl)-phenyl]-(4-fluoro-2-methyl-phenyl)-carbamoyloxy]-ethyl ester (compound 147)

The reaction, work up and purification was conducted as described in the preparation of compound 111. Starting compounds were compound 301 (920 mg, 2.0 mmol) and tetrabutylammonium nicotinate (1.09 mg, 3.0 mmol).

¹³C NMR (CDCl₃): δ 196.5, 163.4, 162.1 (d), 154.0, 151.0, 144.2, 139.3, 138.6, 137.3, 137.1, 135.7, 134.8 (d), 132.7, 131.9, 131.8, 131.0, 130.7, 125.5, 125.2, 124.9, 123.4, 121.3, 118.1 (d), 114.4 (d), 91.1, 21.0, 19.6, 17.9

### Example 48

### Nicotinic acid 1-[[3-chloro-4-(4-chloro-2-methyl-benzoyl)-phenyl]-(4-fluoro-2-methyl-phenyl)-carbamoyloxy]-ethyl ester (compound 148)

The reaction, work up and purification was conducted as described in the preparation of compound 111. Starting compounds were compound 308 (740 mg, 1.5 mmol) and tetrabutylammonium nicotinate (860 mg, 2.4 mmol).

¹³C NMR (CDCl₃): δ 195.4, 163.4, 162.2 (d), 154.0, 151.9, 151.0, 144.4, 141.3, 138.6, 138.0, 137.3, 135.6, 135.2, 134.7 (d), 132.7, 132.3, 131.8, 130.6, 125.8, 125.2, 124.8, 123.4, 121.3, 118.2 (d), 114.4 (d), 91.2, 20.9, 19.6, 17.8

### Example 49

### 2-Hydroxy-benzoic acid 1-[[3-chloro-4-(2-methyl-benzoyl)-phenyl]-(4-fluoro-2-methyl-phenyl)-carbamoyloxy]-ethyl ester (compound 149)

The reaction and work up was conducted as described in the preparation of compound 111 except that the reaction was stopped after 2 days. Starting compounds were compound 301 (920 mg, 2.0 mmol) and tetrabutylammonium 2-hydroxy-benzoate (1.14 g, 3.0 mmol). The crude product was purified by flash chromatography using diethyl ether/petroleum ether as a gradient from 5:95 to 1:1 as the eluent to afford the title compound.

¹³C NMR (CDCl₃): δ 196.5, 168.4, 162.2, 162.1 (d), 151.9, 144.1, 139.3, 138.7, 137.1, 136.4, 135.7, 134.8 (d), 132.7, 131.9, 131.8, 131.0, 130.7, 130.5 (d), 129.9, 125.5, 124.9, 121.3, 119.3, 118.1 (d), 117.8, 114.4 (d), 111.5, 90.8, 21.0, 19.6, 17.8

### Example 50

### Hydroxy-phenyl-acetic acid 1-[[3-chloro-4-(2-methyl-benzoyl)-phenyl]-(4-fluoro-2-methyl-phenyl)-carbamoyloxy]-ethyl ester (compound 150)

The reaction and work up was conducted as described in the preparation of compound 111 except that the reaction was stopped after 2 days. Starting compounds were compound 301 (920 mg, 2.0 mmol) and tetrabutylammonium 1-hydroxyl-1-phenyl-acetate (1.12 g, 3.0 mmol). The crude product was purified by flash chromatography using diethyl ether/petroleum ether as a gradient from 1:9 to 1:1 as the eluent to afford the title compound.

¹³C NMR (CDCl₃): δ 196.5, 171.8, 162.2 (d), 151.7, 144.1, 139.3, 138.6, 137.6, 137.1, 135.8, 134.7 (d), 132.7, 132.0, 131.8, 131.0, 130.7, 130.5, 128.7, 126.6, 125.6, 124.9, 121.3, 118.1 (d), 114.4 (d), 91.5, 72.9, 21.0, 19.2, 17.8

### Example 51

### (S)-2-tert-Butoxycarbonylamino-3-hydroxy-propionic acid 1-[[3-chloro-4-(2-methyl-benzoyl)-phenyl]-(4-fluoro-2-methyl-phenyl)-carbamoyloxy]-ethyl ester (compound 151)(diastereomer A)

The reaction, work up and purification was conducted as described in the preparation of compound 111. Starting compounds were compound 301 (460 mg, 1.0 mmol) and tetrabutylammonium (S)-2-tert-butoxycarbonylamino-3-hydroxy-propionate (550 mg, 1.25 mmol).

¹H NMR (CDCl₃): δ 7.55-7.00 (m, 11H), 6.81 (q, 1H), 4.89 (m, 1H), 4.05 (m, 1H), 3.58 (m, 2H), 2.44 (s, 3H), 2.14 (bs, 3H), 1.37 (bs, 9H), 1.34 (bd, 3H)

### Example 52

### (S)-2-tert-Butoxycarbonylamino-3-hydroxy-propionic acid 1-[[3-chloro-4-(2-methyl-benzoyl)-phenyl]-(4-fluoro-2-methyl-phenyl)-carbamoyloxy]-ethyl ester (compound 152) (diastereomer B)

The title compound was obtained in the above synthesis of compound 151.

¹H NMR (CDCl₃): δ 7.45-7.25 (m, 5H), 7.23-7.08 (m, 3H), 7.07-6.94 (m, 2H), 6.91 (q, 1H), 5.40 (bd, 1H), 4.33 (m, 1H), 4.00 (m, 1H), 3.85 (m, 1H), 2.53 (s, 3H), 2.32 (bs, 1H), 2.27-2.08 (bs, 3H), 1.50-1.40 (bd, 3H), 1.45 (s, 9H)

### Example 53

### Cream Formulation Containing Compound 112

Butyric acid 1-[[3-chloro-4-(2-methyl-benzoyl)-phenyl]-(4-fluoro-2-methyl-phenyl)-carbamoyloxy]-ethyl ester (compound 112, 10 g) was dissolved in diethylenglycolmonoethylether (350 g) and distilled water (350 g) was added. Methylparaben (1 g) and propylparaben (0.2 g) were dissolved in phenoxyethanol (6 g). This solution was mixed with the former solution of Compound 101. Paraffin oil (183 g), cetostearylic alcohol (50 g) and ARLACEL® (50 g) was melted in a vessel at 70 to 80° C. The mixed solutions were likewise heated to 60-70° C. and slowly added to the melted oil phase under high speed stirring. The homogenised components were cooled to room temperature.

### Example 54

EXAMPLE 54
Tablet containing compound 112.

| | |
|---|---|
| Compound 112 (active substance) | 50 mg |
| Lactose | 125 mg |
| Starch | 12 mg |
| Methyl cellulose | 2 mg |
| Sodium carboxymethyl cellulose | 10 mg |
| Magnesium stearate | 1 mg |

The active substance, lactose and starch are mixed to a homogeneous state in a suitable mixer and moistened with a 5 percent aqueous solution of methyl cellulose 15 cps. The mixing is continued until granules are formed. If necessary, the wet granulation is passed through a suitable screen and dried to a water content of less than 1% in a suitable drier, e.g. fluid bed or drying oven. The dried granules are passed through a 1 mm screen and mixed to a homogeneous state with sodium carboxymethyl cellulose. Magnesium stearate is added, and the mixing is continued for a short period of time. Tablets with a weight of 200 mg are produced from the granulation by means of a suitable tabletting machine.

### Example 55

EXAMPLE 55
Formulation for injection containing compound 112.

| | |
|---|---|
| Compound 112 (active substance) | 1% |
| Sodium chloride | q.s. |
| Ethanol | 10% |
| Water for injection to make | 100% |

The active substance is dissolved in ethanol (10%) then water for injection made isotonic with sodium chloride is added to make 100%. The mixture is filled into ampoules and sterilised.

### Example 56

### Cream Formulation Containing Compound 112

Compound 112 (10 g) was dissolved in Octyldodecyl myristate (250g) to form Part A. Methylparaben (1 g) and propylparaben (0.2 g) were dissolved in phenoxyethanol (6 g) and mixed with a 0.025 M Phosphate buffer pH=7.5 (632.8 g) to form Part B. Cetostearyl alcohol (50 g) and ARLACEL 165® (50 g) was melted in a vessel at 70° to 80° C. Part A was added and heated to 60-70° C. The aqueous phase were likewise heated to 60-70° C. and slowly added to the melted oil phase under high speed stirring. The homogenised components were cooled to room temperature.

### Example 57

### Cream Formulation Containing Compound 112—Pemulen Based

Compound 112 (10 g) was dissolved in Octyldodecyl myristate (250 g) and sorbitan oleate (3 g) was added to form Part A. Pemulen TR-2 (3 g) and Carbopol 980 (3 g) were dispersed in Part A in order to break-up any soft agglomerates. Methylparaben (1 g) and propylparaben (0.2 g) were dissolved in phenoxyethanol (6 g) and mixed with water (700 g) to form Part B. With moderate agitation Part B was added to Part A and mix 30-40 minutes or until a smooth dispersion is apparent. Add as much Sodium hydroxide in order to obtain a pH of 7.5 and mix vigorously until a smooth product is obtained. Add water to a final volume of 1000 g.

### Example 58

### Gel Suspension Containing Compound 112

Carbopol 980 (10 g) is dispersed in water (400 g) and neutralised with a sodium hydroxide (10%) to pH=7.5 (Part A). In order to prepare Part B, Methylparaben (1 g) and propylparaben (0.2 g) were dissolved in phenoxyethanol (6 g). Methylcellulose (10 g) is dispersed in cold water (100 g) and hot water is added (300 g), which is termed Part C. Part B and Part C is thoroughly mixed and micronized. Compound 101 (10 g) is dispersed in the combined mixture (Part D). Part D is added to the neutralised gel under mild agitation. Water is added to make a final weight of 1000 gram, the water is thoroughly mixed into the thickened gel using mild agitation.

### Example 59

### Gel Formulation Containing Compound 112

Carbopol 980 (10 g) and Aerosil R 972 2% is dispersed in water (600 g) and neutralised with a 10% sodium hydroxide solution to pH=7.5 (Part A). In order to prepare Part B, Methylparaben (1 g) and propylparaben (0.2 g) were dissolved in phenoxyethanol (6 g). Compound 112 (10 g) is dissolved in Labrasol (300 g) (Part C). Part B and Part C is combined to form Part D, which is then added to the neutralised gel under mild agitation. Water is added to make a final weight of 1000 g; the water is thoroughly mixed into the thickened gel using mild agitation.

### Example 60

### Ointment Formulation Containing Compound 112

Compound 112 (5 g) is dissolved in Octyldodecyl myristate (500 g) to form Part A. Aerosil R 972 (70 g) is then dispersed into Part A by low speed agitation to form part B. Part B is then combined with Vaseline (380 g).

### Example 61

### Lotion with Ethanol Containing Compound 112

Compound 112 (5 g) is dissolved in Ethanol (500 g) to form Part A. Polyethylene glycol 300 is then dispersed into Part A by low speed agitation.

### Example 62

### Lotion with Ethanol Containing Compound 112

Compound 112 (15 g) is dissolved in Ethanol (600 g) and Octyldodecyl myristate (100 g) and Water (300 g) is then added to form Part A. Hydroxypropylmethylcellulose is dispersed into Part A by low speed agitation.

1. A compound of general formula I 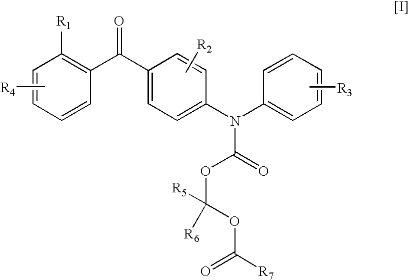
wherein R₁ represents a substituent selected from the group consisting of halogen, hydroxy, mercapto, trifluoromethyl, amino, (C₁-C₃)alkyl, (C₂-C₃)olefinic group, (C₁-C₃)alkoxy, (C₁-C₃)alkylthio, (C₁-C₄)alkylamino and cyano;
R₂ represents one or more, same or different substituents selected from the group consisting of hydrogen, halogen, hydroxy, mercapto, trifluoromethyl, amino, (C₁-C₃)alkyl, (C₂-C₃)olefinic group, (C₁-C₃)alkoxy, (C₁-C₃)alkylthio, (C₁-C₄)alkylamino, (C₁-C₃)alkoxycarbonyl, cyano, and nitro;
R₃ represents one or more, same or different substituents selected from the group consisting of hydrogen, halogen, hydroxy, mercapto, trifluoromethyl, cyano, carboxy, carbamoyl, (C₁-C₄)alkyl, (C₂-C₄)olefinic group, (C₁-C₄)alkoxy, (C₁-C₄)alkylthio, and (C₁-C₄)alkoxycarbonyl;
R₄ represents one or more, same or different substituents selected from the group consisting of hydrogen, halogen, hydroxy, mercapto, trifluoromethyl, amino, (C₁-C₃)alkyl, (C₂-C₃)olefinic group, (C₁-C₃)alkoxy, (C₁-C₃)alkylthio, (C₁-C₄)alkylamino, (C₁-C₃)alkoxycarbonyl, cyano, and nitro;
R₅ represents hydrogen, (C₁-C₆)alkyl and (C₂-C₆)olefinic group;
R₆ represents hydrogen, (C₁-C₆)alkyl and (C₂-C₆)olefinic group;
R₇ represents (C₁-C₁₈)alkyl, (C₃-C₈)cyclic hydrocarbon group, (C₂-C₁₈)olefinic group, heterocyclyl, (C₂-C₁₈)alkynyl, (C₁-C₁₈)alkyl-heterocyclyl, (C₁-C₁₈)alkyl-(C₃-C₈)cyclic hydrocarbon group, (C₂-C₁₈)olefinic group-heterocyclyl, (C₂-C₁₈)olefinic group-(C₃-C₈)cyclic hydrocarbon group, (C₂-C₁₈)alkynyl-heterocyclyl, (C₂-C₁₈)alkynyl-(C₃-C₈)cyclic hydrocarbon group; and wherein R₇ may optionally be substituted by one or more substituents represented by R₈;
R₈ represents halogen, hydroxy, mercapto, trifluoromethyl, amino, (C₁-C₆)alkyl, (C₁-C₆)alkoxy, (C₁-C₆)alkylthio, (C₁-C₆)alkylamino, (C₁-C₆)alkoxycarbonyl, (C₁-C₉)trialkylammonium in association with a pharmaceutically acceptable anion, (C₂-C₁₂)dialkylphosphinoyl, (C₁-C₆)alkyl(hydroxy)phosphinoyl, (C₂-C₁₂)dialkylphosphinoyloxy, (C₁-C₆)alkyl(hydroxy)phosphinoyloxy, dihydroxyphosphinoyl, dihydroxyphosphinoyloxy, cyano, azido, nitro, —CHO, —COOH, —CONH₂, —CONHR′, —CONRR′ wherein R and R′ represent (C₁-C₃)alkyl or Y—R₉;
Y represents —O—, —S—, —S(O)—, —S(O)₂—, —NRₐ—, —NRₐC(O)NR_{b}—, —NRₐC(O)—, —C(O)NRₐ—, —C(O)—, —C(O)O—, —OC(O)—, —NRₐC(O)O—, —OC(O)NRₐ—, —S(O)₂NRₐ—, —NRₐS(O)₂—, —OC(O)O— or —O(CH₂CH₂O)ₙ— wherein n is an integer between 1 and 6, and Rₐ and R_{b} independently represents hydrogen or (C₁-C₃)alkyl;
R₉ represents (C₁-C₆)alkyl, (C₂-C₆)olefinic group, (C₃-C₆)cyclic hydrocarbon group, heterocyclyl, (C₂-C₆)alkynyl, (C₁-C₆)alkyl-(C₃-C₆)cyclic hydrocarbon or (C₁-C₆)alkyl-heterocyclyl, and wherein R₉ may optionally be substituted by one or more substituents represented by R₁₀;
R₁₀ represents halogen, hydroxy, mercapto, trifluoromethyl, amino, (C₁-C₆)alkyl, (C₁-C₆)alkoxy, (C₁-C₆)alkylthio, (C₁-C₆)alkylamino or (C₁-C₆)alkoxycarbonyl;
and pharmaceutically acceptable salts, solvates and hydrates thereof.

2. A compound according to claim 1, wherein R₁ represents fluoro, chloro or bromo, methyl or methoxy

3. A compound according to claim 1 or 2, wherein R₂ represents on or more substituents selected from the list consisting of hydrogen, fluoro, chloro, methyl or methoxy.

4. A compound according to claim 1, wherein R₂ represents 2-chloro.

5. A compound according to 1-4 claim 1, wherein R₃ represents one or more substituents selected from the list consisting of hydrogen, fluoro, chloro, methyl, ethyl, ethenyl or methoxy.

6. A compound according to claim 1, wherein R₃ represents 2-methyl and 4-fluoro, or 2-methyl and 4-bromo.

7. A compound according to claim 1, wherein R₄ represents one or more substituents selected from the list consisting of hydrogen, fluoro, chloro, bromo, methyl and methoxy.

8. A compound according to claim 1, wherein, R₄ represents 4-chloro.

9. A compound according to claim 1, wherein R₅ and R₆ each independently represent hydrogen or (C₁-C₆)alkyl.

10. A compound according to claim 1, wherein R₅ or R₆ each independently represents hydrogen, (C₁-C₄)alkyl or methyl.

11. A compound according to claim 1, wherein R₇ represents
(C₁-C₁₀)alkyl, (C₃-C₆)cyclic hydrocarbon group, (C₂-C₁₀)olefinic group, heterocyclyl, (C₂-C₁₀)alkynyl, (C₁-C₁₀)alkyl-heterocyclyl, (C₁-C₁₀)alkyl-(C₃-C₆)cyclic hydrocarbon group, (C₂-C₁₀)olefinic group-heterocyclyl, (C₂-C₁₀), olefinic group-(C₃-C₆)cyclic hydrocarbon group, (C₂-C₁₀)alkynyl-heterocyclyl, (C₂-C₁₀)alkynyl-(C₃-C₆)cyclic hydrocarbon group; and wherein R₇ may optionally be substituted by one or more substituents represented by R₈.

12. A compound according to claim 1, wherein R₇ represents
(C₁-C₆)alkyl, (C₃-C₆)cyclic hydrocarbon group, (C₂-C₆)olefinic group, heterocyclyl, (C₂-C₆)alkynyl, (C₁-C₆)alkyl-heterocyclyl, (C₁-C₆)alkyl-(C₃-C₆)cyclic hydrocarbon group, (C₂-C₆)olefinic group-heterocyclyl, (C₂-C₆), olefinic group-(C₃-C₆)cyclic hydrocarbon group, (C₂-C₆)alkynyl-heterocyclyl, (C₂-C₆)alkynyl-(C₃-C₆)cyclic hydrocarbon group; and wherein R₇ may optionally be substituted by one or more substituents represented by R₈.

13. A compound according to claim 1, wherein R₇ represents methyl, ethyl, propyl, iso-propyl, butyl, tert-butyl, pentyl, heptyl, nonyl, 2-methyl-propyl, 1-methyl-propyl, 2,2-dimethyl-propyl, cyclopropyl, cyclobutyl, phenyl, ethenyl, propenyl, phenylmethyl, phenyl-1-allyl or 2-, 3- or 4-pyridyl, all of which may be substituted by R₈.

14. A compound according to claim 1, wherein R₈ represents halogen, hydroxy, trifluoromethyl, amino, (C₁-C₆)alkyl, (C₁-C₆)alkoxy, (C₁-C₆)alkylamino, (C₁-C₆)alkoxycarbonyl, (C₁-C₉)trialkylammonium in association with a pharmaceutically acceptable anion, cyano, COOH or Y—R₉.

15. A compound according to claim 1, wherein R₈ represents hydroxyl or carboxy.

16. A compound according to claim 1, wherein Y represents —O—, —NRₐ—, —NRₐC(O)—, —C(O)NRₐ—, —C(O)—, —C(O)O—, —OC(O)—, —NRₐC(O)O— or —O(CH₂CH₂O)ₙ— wherein n is 1, 2, 3 or 4, and Rₐ and R_{b} both represents hydrogen.

17. A compound according to claim 1, wherein Y represents —C(O)—O—, NH—C(O)—O—, —O—, —O—C(O)— or —O(CH₂CH₂O)ₙ— wherein n is 3.

18. A compound according to claim 1, wherein R₉ represents
(C₁-C₄)alkyl, (C₂-C₃)olefinic group, (C₃-C₆)cyclic hydrocarbon group, heterocyclyl, (C₂-C₃)alkynyl, (C₁-C₃)alkyl-(C₃-C₆)cyclic hydrocarbon or (C₁-C₃)alkyl-heterocyclyl, wherein R₉ may optionally be substituted by one or more substituents represented by R₁₀.

19. A compound according to claim 1, wherein R₉ represents
(C₁-C₄)alkyl or (C₁-C₃)alkyl-(C₃-C₆)cyclic hydrocarbon.

20. A compound according to claim 1, wherein R₉ represents methyl, ethyl, tert-butyl or phenylmethyl.

21. A compound according to claim 1, wherein R₁₀ represents fluoro, chloro, hydroxy, trifluoromethyl, amino, (C₁-C₃)alkyl, (C₁-C₃)alkoxy, (C₁-C₃)alkylamino or (C₁-C₃)alkoxycarbonyl.

22. A compound according to claim 1, wherein R₁ is methyl; R₂ is 2-chloro; R₃ is 2-methyl and 4-fluoro, or 2-methyl and 4-bromo; R₄ is hydrogen or 4-chloro;
R₅ and R₆ independently represent hydrogen or (C₁-C₄)alkyl;
R₇represents (C₁-C₁₀)alkyl, (C₃-C₆)cyclic hydrocarbon group, (C₂-C₁₀)olefinic group, heterocyclyl, (C₂-C₁₀)alkynyl, (C₁-C₁₀)alkyl-heterocyclyl, (C₁-C₁₀)alkyl-(C₃-C₆)cyclic hydrocarbon group, (C₂-C₁₀)olefinic group-heterocyclyl, (C₂-C₁₀), olefinic group-(C₃-C₆)cyclic hydrocarbon group, (C₂-C₁₀)alkynyl-heterocyclyl, (C₂-C₁₀)alkynyl-(C₃-C₆)cyclic hydrocarbon group; and wherein R₇ may optionally be substituted by one or more substituents represented by R₈;
R₈ represents halogen, hydroxy, trifluoromethyl, amino, (C₁-C₆)alkyl, (C₁-C₆)alkoxy, (C₁-C₆)alkylamino, (C₁-C₆)alkoxycarbonyl, (C₁-C₉)trialkylammonium in association with a pharmaceutically acceptable anion, cyano, —COOH or Y—R₉;
Y represents —O—, —NRₐ—, —NRₐC(O)—, —C(O)NRₐ—, —C(O)—, —C(O)O—, —OC(O)—, —NRₐC(O)O— or —O(CH₂CH₂O)ₙ— wherein n is 1, 2, 3 or 4, and Rₐ and R_{b} both represents hydrogen;
R₉ represents (C₁-C₃)alkyl, (C₂-C₃)olefinic group, (C₃-C₆)cyclic hydrocarbon group, heterocyclyl, (C₂-C₃)alkynyl, (C₁-C₃)alkyl-(C₃-C₆)cyclic hydrocarbon or (C₁-C₃)alkyl-heterocyclyl, wherein R₉ may optionally be substituted by one or more substituents represented by R₁₀;
R₁₀represents fluoro, chloro, hydroxy, trifluoromethyl, amino, (C₁-C₃)alkyl, (C₁-C₃)alkoxy, (C₁-C₃)alkylamino or (C₁-C₃)alkoxycarbonyl;
and pharmaceutically acceptable salts solvates or hydrates thereof.

23. A compound according to claim 1, wherein R₁ is methyl; R₂ is 2-chloro; R₃ is 2-methyl and 4-fluoro, or 2-methyl and 4-bromo; R₄ is hydrogen or 4-chloro;
R₅ and R₆ independently represent hydrogen or (C₁-C₄)alkyl;
R₇ represents (C₁-C₆)alkyl, (C₃-C₆)cyclic hydrocarbon group, (C₂-C₆)olefinic group, heterocyclyl, (C₂-C₆)alkynyl, (C₁-C₆)alkyl-heterocyclyl, (C₁-C₆)alkyl-(C₃-C₆)cyclic hydrocarbon group, (C₂-C₆)olefinic group-heterocyclyl, (C₂-C₆), olefinic group-(C₃-C₆)cyclic hydrocarbon group, (C₂-C₆)alkynyl-heterocyclyl, (C₂-C₆)alkynyl-(C₃-C₆)cyclic hydrocarbon group; and wherein R₇ may optionally be substituted by one or more substituents represented by R₈;
R₈represents halogen, hydroxy, trifluoromethyl, amino, (C₁-C₆)alkyl, (C₁-C₆)alkoxy, (C₁-C₆)alkylamino, (C₁-C₆)alkoxycarbonyl, (C₁-C₉)trialkylammonium in association with a pharmaceutically acceptable anion, cyano, —COOH or Y—R₉;
Y represents —O—, —NRₐ—, —NRₐC(O)—, —C(O)NRₐ—, —C(O)—, —C(O)O—, —OC(O)—, —NRₐC(O)O— or —O(CH₂CH₂O)ₙ— wherein n is 1, 2, 3 or 4, and Rₐ and R_{b} both represents hydrogen;
R₉ represents (C₁-C₃)alkyl, (C₂-C₃)olefinic group, (C₃-C₆)cyclic hydrocarbon group, heterocyclyl, (C₂-C₃)alkynyl, (C₁-C₃)alkyl-(C₃-C₆)cyclic hydrocarbon or (C₁-C₃)alkyl-heterocyclyl, wherein R₉ may optionally be substituted by one or more substituents represented by R₁₀;
R₁₀represents fluoro, chloro, hydroxy, trifluoromethyl, amino, (C₁-C₃)alkyl, (C₁-C₃)alkoxy, (C₁-C₃)alkylamino or (C₁-C₃)alkoxycarbonyl;
and pharmaceutically acceptable salts solvates or hydrates thereof.

24. A compound according to claim 1, wherein R₁ is methyl; R₂ is 2-chloro; R₃ is 2-methyl and 4-fluoro, or 2-methyl and 4-bromo; R₄ is hydrogen or 4-chloro;
R₅ and R₆ independently represent hydrogen or methyl;
R₇ represents methyl, ethyl, propyl, iso-propyl, butyl, tert-butyl, pentyl, heptyl, nonyl, 2-methyl-propyl, 1-methyl-propyl, 2,2-dimethyl-propyl, cyclopropyl, cyclobutyl, phenyl, ethenyl, propenyl, phenylmethyl, phenyl-1-allyl or 2-, 3- or 4-pyridyl, all of which may be substituted by R₈;
R₈ represents hydroxyl, carboxy;
Y represents —C(O)—O—, NH—C(O)—O, —O—, —O—C(O)— or —O(CH₂—CH₂—O)ₙ—, n being 3;
R₉ represents methyl, ethyl, tert-butyl or phenylmethyl;
R₁₀ represents fluoro, chloro, hydroxy, trifluoromethyl, amino, (C₁-C₃)alkyl, (C₁-C₃)alkoxy, (C₁-C₃)alkylamino or (C₁-C₃)alkoxycarbonyl;
and pharmaceutically acceptable salts, solvates and hydrates thereof.

25. A compound according to claim 1 selected from the group consisting of
Succinic acid benzyl ester 1-[[3-chloro-4-(2-methyl-benzoyl)-phenyl]-(4-fluoro-2-methyl-phenyl)-carbamoyloxy]-ethyl ester;
Succinic acid mono-{1-[[3-chloro-4-(2-methyl-benzoyl)-phenyl]-(4-fluoro-2-methyl-phenyl)-carbamoyloxy]-ethyl}ester;
Sodium 3-{1-[[3-chloro-4-(2-methyl-benzoyl)-phenyl]-(4-fluoro-2-methyl-phenyl)-carbamoyloxy]-ethoxycarbonyl}-propionate;
{2-[2-(2-Methoxy-ethoxy)-ethoxy]-ethoxy}-acetic acid 1-[[3-chloro-4-(2-methyl-benzoyl)-phenyl]-(4-fluoro-2-methyl-phenyl)-carbamoyloxy]-ethyl ester;
{2-[2-(2-Methoxy-ethoxy)-ethoxy]-ethoxy}-acetic acid 1-{(4-bromo-2-methyl-phenyl)-[3-chloro-4-(2-methyl-benzoyl)-phenyl]-carbamoyloxy}-ethyl ester;
Succinic acid benzyl ester 1-{(4-bromo-2-methyl-phenyl)-[3-chloro-4-(2-methyl-benzoyl)-phenyl]-carbamoyloxy}-ethyl ester;
Succinic acid mono-(1-{(4-bromo-2-methyl-phenyl)-[3-chloro-4-(2-methyl-benzoyl)-phenyl]-carbamoyloxy}-ethyl) ester;
Succinic acid {(4-bromo-2-methyl-phenyl)-[3-chloro-4-(2-methyl-benzoyl)-phenyl]-carbamoyloxy}-methyl ester methyl ester;
Succinic acid benzyl ester {(4-bromo-2-methyl-phenyl)-[3-chloro-4-(2-methyl-benzoyl)-phenyl]-carbamoyloxy}-methyl ester;
Acetic acid 1-[[3-chloro-4-(2-methyl-benzoyl)-phenyl]-(4-fluoro-2-methyl-phenyl)-carbamoyloxy]-ethyl ester;
Propionic acid 1-[[3-chloro-4-(2-methyl-benzoyl)-phenyl]-(4-fluoro-2-methyl-phenyl)-carbamoyloxy]-ethyl ester;
Butyric acid 1-[[3-chloro-4-(2-methyl-benzoyl)-phenyl]-(4-fluoro-2-methyl-phenyl)-carbamoyloxy]-ethyl ester;
Butyric acid [[3-chloro-4-(2-methyl-benzoyl)-phenyl]-(4-fluoro-2-methyl-phenyl)-carbamoyloxy]-methyl ester;
Pentanoic acid 1-[[3-chloro-4-(2-methyl-benzoyl)-phenyl]-(4-fluoro-2-methyl-phenyl)-carbamoyloxy]-ethyl ester;
Hexanoic acid 1-[[3-chloro-4-(2-methyl-benzoyl)-phenyl]-(4-fluoro-2-methyl-phenyl)-carbamoyloxy]-ethyl ester;
Octanoic acid 1-[[3-chloro-4-(2-methyl-benzoyl)-phenyl]-(4-fluoro-2-methyl-phenyl)-carbamoyloxy]-ethyl ester;
Decanoic acid 1-[[3-chloro-4-(2-methyl-benzoyl)-phenyl]-(4-fluoro-2-methyl-phenyl)-carbamoyloxy]-ethyl ester;
Succinic acid 1-[[3-chloro-4-(2-methyl-benzoyl)-phenyl]-(4-fluoro-2-methyl-phenyl)-carbamoyloxy]-ethyl ester ethyl ester;
Methoxy-acetic acid 1-[[3-chloro-4-(2-methyl-benzoyl)-phenyl]-(4-fluoro-2-methyl-phenyl)-carbamoyloxy]-ethyl ester;
Methoxy-acetic acid [[3-chloro-4-(2-methyl-benzoyl)-phenyl]-(4-fluoro-2-methyl-phenyl)-carbamoyloxy]-methyl ester;
Butyric acid 1-[[3-chloro-4-(4-chloro-2-methyl-benzoyl)-phenyl]-(4-fluoro-2-methyl-phenyl)-carbamoyloxy]-ethyl ester;
3-Methoxy-propionic acid 1-[[3-chloro-4-(4-chloro-2-methyl-benzoyl)-phenyl]-(4-fluoro-2-methyl-phenyl)-carbamoyloxy]-ethyl ester;
3,3-Dimethyl-butyric acid [[3-chloro-4-(2-methyl-benzoyl)-phenyl]-(4-fluoro-2-methyl-phenyl)-carbamoyloxy]-methyl ester;
Cyclopropanecarboxylic acid [[3-chloro-4-(2-methyl-benzoyl)-phenyl]-(4-fluoro-2-methyl-phenyl)-carbamoyloxy]-methyl ester;
Cyclobutanecarboxylic acid [[3-chloro-4-(2-methyl-benzoyl)-phenyl]-(4-fluoro-2-methyl-phenyl)-carbamoyloxy]-methyl ester;
2-Hydroxy-propionic acid 1-[[3-chloro-4-(2-methyl-benzoyl)-phenyl]-(4-fluoro-2-methyl-phenyl)-carbamoyloxy]-ethyl ester;
2-Methyl-but-2-enoic acid 1-[[3-chloro-4-(2-methyl-benzoyl)-phenyl]-(4-fluoro-2-methyl-phenyl)-carbamoyloxy]-ethyl ester;
2-Hydroxy-2-methyl-propionic acid 1-[[3-chloro-4-(2-methyl-benzoyl)-phenyl]-(4-fluoro-2-methyl-phenyl)-carbamoyloxy]-ethyl ester;
2-Hydroxy-2-methyl-propionic acid 1-[[3-chloro-4-(4-chloro-2-methyl-benzoyl)-phenyl]-(4-fluoro-2-methyl-phenyl)-carbamoyloxy]-ethyl ester;
Isobutyric acid 1-[[3-chloro-4-(2-methyl-benzoyl)-phenyl]-(4-fluoro-2-methyl-phenyl)-carbamoyloxy]-ethyl ester;
Isobutyric acid [[3-chloro-4-(2-methyl-benzoyl)-phenyl]-(4-fluoro-2-methyl-phenyl)-carbamoyloxy]-methyl ester;
2,2-Dimethyl-propionic acid 1-[[3-chloro-4-(2-methyl-benzoyl)-phenyl]-(4-fluoro-2-methyl-phenyl)-carbamoyloxy]-ethyl ester;
3-Methyl-butyric acid 1-[[3-chloro-4-(2-methyl-benzoyl)-phenyl]-(4-fluoro-2-methyl-phenyl)-carbamoyloxy]-ethyl ester;
2-Methyl-butyric acid 1-[[3-chloro-4-(2-methyl-benzoyl)-phenyl]-(4-fluoro-2-methyl-phenyl)-carbamoyloxy]-ethyl ester;
Cyclopropanecarboxylic acid 1-[[3-chloro-4-(2-methyl-benzoyl)-phenyl]-(4-fluoro-2-methyl-phenyl)-carbamoyloxy]-ethyl ester;
Acrylic acid 1-[[3-chloro-4-(2-methyl-benzoyl)-phenyl]-(4-fluoro-2-methyl-phenyl)-carbamoyloxy]-ethyl ester;
But-2-enoic acid 1-[[3-chloro-4-(2-methyl-benzoyl)-phenyl]-(4-fluoro-2-methyl-phenyl)-carbamoyloxy]-ethyl ester;
But-2-enoic acid [[3-chloro-4-(2-methyl-benzoyl)-phenyl]-(4-fluoro-2-methyl-phenyl)-carbamoyloxy]-methyl ester;
Cyclobutanecarboxylic acid 1-[[3-chloro-4-(2-methyl-benzoyl)-phenyl]-(4-fluoro-2-methyl-phenyl)-carbamoyloxy]-ethyl ester;
3-Methoxy-propionic acid 1-[[3-chloro-4-(2-methyl-benzoyl)-phenyl]-(4-fluoro-2-methyl-phenyl)-carbamoyloxy]-ethyl ester;
2-Acetoxy-propionic acid 1-[[3-chloro-4-(2-methyl-benzoyl)-phenyl]-(4-fluoro-2-methyl-phenyl)-carbamoyloxy]-ethyl ester;
2,2-Dimethyl-propionic acid [[3-chloro-4-(2-methyl-benzoyl)-phenyl]-(4-fluoro-2-methyl-phenyl)-carbamoyloxy]-methyl ester;
3-Phenyl-acrylic acid 1-[[3-chloro-4-(2-methyl-benzoyl)-phenyl]-(4-fluoro-2-methyl-phenyl)-carbamoyloxy]-ethyl ester;
Benzoic acid 1-[[3-chloro-4-(2-methyl-benzoyl)-phenyl]-(4-fluoro-2-methyl-phenyl)-carbamoyloxy]-ethyl ester;
Pyridine-2-carboxylic acid 1-[[3-chloro-4-(2-methyl-benzoyl)-phenyl]-(4-fluoro-2-methyl-phenyl)-carbamoyloxy]-ethyl ester;
Isonicotinic acid 1-[[3-chloro-4-(2-methyl-benzoyl)-phenyl]-(4-fluoro-2-methyl-phenyl)-carbamoyloxy]-ethyl ester;
Nicotinic acid 1-[[3-chloro-4-(2-methyl-benzoyl)-phenyl]-(4-fluoro-2-methyl-phenyl)-carbamoyloxy]-ethyl ester;
Nicotinic acid 1-[[3-chloro-4-(4-chloro-2-methyl-benzoyl)-phenyl]-(4-fluoro-2-methyl-phenyl)-carbamoyloxy]-ethyl ester;
2-Hydroxy-benzoic acid 1-[[3-chloro-4-(2-methyl-benzoyl)-phenyl]-(4-fluoro-2-methyl-phenyl)-carbamoyloxy]-ethyl ester;
Hydroxy-phenyl-acetic acid 1-[[3-chloro-4-(2-methyl-benzoyl)-phenyl]-(4-fluoro-2-methyl-phenyl)-carbamoyloxy]-ethyl ester;
(S)-2-tert-Butoxycarbonylamino-3-hydroxy-propionic acid 1-[[3-chloro-4-(2-methyl-benzoyl)-phenyl]-(4-fluoro-2-methyl-phenyl)-carbamoyloxy]-ethyl ester (diastereomer A); and
(S)-2-tert-Butoxycarbonylamino-3-hydroxy-propionic acid 1-[[3-chloro-4-(2-methyl-benzoyl)-phenyl]-(4-fluoro-2-methyl-phenyl)-carbamoyloxy]-ethyl ester (diastereomer B).

26. A compound according to claim 1 for use in therapy.

27. A pharmaceutical composition comprising a compound according to claim 1, optionally together with another therapeutically active compound, and one or more pharmaceutically acceptable carriers or excipients.

28. A formulation according to claim 27, wherein said other therapeutically active compound is selected from the list consisting of glucocorticoids, vitamin D analogues, anti-histamines, platelet activating factor (PAF) antagonists, anticolinergic agents, methyl xanthines, β-adrenergic agents, COX-2 inhibitors, salicylates, indomethacin, flufenamate, naproxen, timegadine, gold salts, penicillamine, serum cholesterol-reducing agents, retinoids, zinc salts, and salicylazosulfapyridin (Salazopyrin).

29. A method for the treatment of acne, atopic dermatitis, contact dermatitis, psoriasis, asthma, allergy, arthritis, rheumatoid arthritis, spondyloarthritis, gout, atherosclerosis, chronic inflammatory bowel disease, uveitis and septic shock, the method comprising administering to a patient in need thereof an effective amount of a compound according to claim 1, optionally in combination with another therapeutically active compound.

30. A method according to claim 29, wherein said other therapeutically active compound is selected from the list consisting of glucocorticoids, vitamin D analogues, anti-histamines, platelet activating factor (PAF) antagonists, anticolinergic agents, methyl xanthines, β-adrenergic agents, COX-2 inhibitors, salicylates, indomethacin, flufenamate, naproxen, timegadine, gold salts, penicillamine, serum cholesterol-reducing agents, retinoids, zinc salts, and salicylazosulfapyridin (Salazopyrin).

31. The use of a compound according to claim 1 in the manufacture of a medicament for the treatment of acne, atopic dermatitis, contact dermatitis, psoriasis, asthma, allergy, arthritis, rheumatoid arthritis, spondyloarthritis, gout, atherosclerosis, chronic inflammatory bowel disease, uveitis or septic shock.
